(19) United States
(12) Patent Application Publication
(10) Pub. No.: US 2020/0061617 A1
(43) Pub. Date: Feb. 27, 2020

(54) DEVICES AND METHODS FOR NUCLEIC ACID IDENTIFICATION

(71) Applicant: Niagara Biosciences, Inc., Worcester, MA (US)

(72) Inventor: Vishal A. Patil, Arlington, MA (US)

(21) Appl. No.: 16/467,347

(22) PCT Filed: Dec. 7, 2017
(86) PCT No.: PCT/US2017/065076

§ 371 Date: Jun. 6, 2019

Related U.S. Application Data
(60) Provisional application No. 62/431,398, filed on Dec. 7, 2016.

(51) Int. Cl. B01L 3/00; C12Q 1/6869; G01N 21/76

(52) U.S. Cl. CPC B01L 3/502761; B01L 3/502776; C12Q 1/6869; B01L 2300/0848; B01L 2200/0636; B01L 2200/0663; B01L 2200/10; G01N 21/76

(57) ABSTRACT

Provided herein are devices and methods for obtaining nucleotide sequence information from nucleic acid and nucleic acid samples. The device includes a microfluidic channel that aids in manipulating a sample as the sample flows through various regions of the channel. The devices and methods are sensitive enough to detect signal from and thus interrogate single nucleic acids on an individual basis rather than as a bulk population.

### CROSS-REFERENCE TO RELATED APPLICATIONS

This application claims the benefit under 35 U.S.C. § 119(e) of U.S. Provisional Application Ser. No. 62/431,398, filed on Dec. 7, 2016, entitled “DEVICES AND METHODS FOR NUCLEIC ACID IDENTIFICATION,” the contents of which are incorporated by reference herein.

### BACKGROUND

Nucleic acid analysis, including nucleic acid sequencing, has enabled a diverse range of applications including detection of microorganisms for medical purposes. Existing nucleic acid technologies for identifying microorganisms include pulse field gel electrophoresis (PFGE), nucleic acid amplification techniques such as polymerase chain reaction (PCR) and including quantitative reverse transcriptase PCR (RT-qPCR), optical mapping, and hybridization based mapping.

In PFGE, a restriction enzyme (also known as a restriction endonuclease, and referred to herein as RE) that cuts genomic DNA rarely (due to the infrequency of its binding or cleavage site) is used to digest genomic DNA. The digested DNA is then separated in a gel matrix using pulsed electric field. The technique is widely used in epidemiological studies of pathogens. As an example, it is used by the Center for Disease Prevention and Control to identify disease outbreaks. Although it is an established methodology that uses universal reagents, it is labor and time intensive, requiring a laborious sample preparation and yielding results within about 2 days. Additionally, strain typing with the accuracy required is not always possible using a single enzyme digestion. PFGE also cannot handle complex mixtures of pathogens (and thus genomic DNA). Finally, the technique typically requires on the order of 1 microgram of DNA, which may be far more DNA than can be obtained from certain samples.

In PCR, a fragment of DNA or a region within a larger fragment of DNA is amplified and the presence or absence of the product or the sequence of the product is used to determine the presence and identity of the pathogen. This method can be used for complex mixtures of pathogens and DNA because the amplification may be selective (i.e., it may only amplify DNA from a particular pathogen and not genomic DNA from others). It is also highly sensitive, and is able to amplify and thus detect very few copies of starting DNA. It is also easily automated and requires easy sample preparation. It does however require preliminary knowledge of the sequence of the target DNA in order to design primers, and it is dependent on the ability to hybridize the primers to the target DNA. Hybridization may vary between primer pairs, causing certain targets to be amplified to a greater extent than others even though they have been present in the original sample in equal amounts.

Optical mapping is a technique that involves fixing elongated and intercalated DNA fragments to a glass slide and digesting the fixed DNA using REs. Maps of the DNA fragments are then obtained by measuring the gaps (or distances) between the digested fragments. This process, like PFGE, is time intensive with the entire process taking about a week. In addition, the technique does not lend itself to high throughput analysis.

Still other methods involve hybridization of probes to DNA fragments. One such method involves linearizing and fixing DNA in nanochannels, followed by hybridizing the fixed DNA to sequence specific probes. The DNA is then scanned to determine the presence and location of the hybridized probes. The requirement for hybridization of the probes can reduce specificity and introduce artefacts into the ultimate readout.

### SUMMARY

Provided herein is a device and corresponding method for nucleic acid analysis that overcomes various limitations of the foregoing methodologies. The approach described herein is able to generate a map or signature of a nucleic acid or a nucleic acid fragment, without the need for hybridization probes, or amplification of the original sample, and without any need for fixation (or immobilization) of the nucleic acid. Significantly, it can be performed in a high throughput manner on the order of minutes, thereby facilitating the analysis of samples at a much higher rate than previously possible.

The device can be used with virtually any nucleic acid (e.g., DNA) sample, and requires minimal manipulation of the sample. Accordingly, the samples can be prepared quickly and easily and the analysis itself can be performed within minutes. For example, sample preparation time may be on the order of about 1 hour and analysis time may be on the order of about 15 minutes. The approach also lends itself to automation. Equally significant, the device can be used with a single molecule analysis technique that can be performed on as little as a few picograms of DNA as the starting material. This could reduce the time it takes to work up a sample, including reducing the amount of culture needed before isolating the DNA. Further advantages will be described or will become apparent herein.

In its broadest sense, the device allows sequence information, and thus maps, of nucleic acids such as DNA, to be obtained by digesting the nucleic acids in a manner such that the digested fragments are analyzed in the order in which they were present in their “parent” nucleic acid and in some instances cleaved from their “parent” nucleic acid. The analysis involves determining the length of the digested fragments, based for example on intercalator brightness. In this way, the readout from the assay is a series of digested fragments each of a particular length (determined by their signal intensity), in an order that is faithful to the order of the fragments in the parent nucleic acid from which they were cleaved. The ability to carry out the analysis, including the entire analysis, in flow (i.e., while the nucleic acid is moving rather than fixed to a solid support) allows for high-throughput analysis.

More specifically, the method involves contacting a parent nucleic acid (such as DNA) with REs of a single type under a condition that allows the RE to bind to but not cleave the nucleic acid (referred to herein as a “binding condition”). An example of such a condition is a solution that contains calcium ions but not magnesium and/or manganese ions (or magnesium and/or manganese ions at a concentration that is insufficient for the RE to cleave the nucleic acid to which it binds or is bound). After incubation with the RE under this condition, the parent nucleic acid is stretched in flow. Stretching of the nucleic acid can be accomplished with the device in a number of ways including by altering sheath fluid flow and/or altering the geometry of the channel through which the parent nucleic acid travels. The nucleic acid can also be stretched by electrophoretic means based on its negative charge. Methods that do not involve charge to stretch and/or move nucleic acids are referred to herein as “non-charge” or “non-electrophoretic” methods.

Once the parent nucleic acid is fully or nearly fully stretched, it is exposed to a condition that allows the RE to cleave the nucleic acid (referred to herein as a “cleaving condition”). An example of a cleaving condition is a solution that contains magnesium and/or manganese ions, which will initiate digestion of the nucleic acid by the bound RE. The cleaving condition can also be an increased concentration of magnesium and/or manganese ions sufficient for the RE to cleave the nucleic acid to which it is bound. These ions can be introduced into the channel of the device through which the parent nucleic acid is travelling for example through the use of porous channel walls or side channels through which sheath fluid and ions travel. In some instances, digestion may occur on single elongated nucleic acids in an ordered manner starting at one end and continuing to the opposite end. In other instances, digestion may occur simultaneously or nearly simultaneously with all the bound RE cutting the parent nucleic acid at the same time or nearly the same time. In still other instances, digestion may occur randomly with bound RE cutting the parent nucleic acid at different times and optionally independently of each other. The parent nucleic acid is maintained in a sufficiently stretched state until digestion is complete (e.g., all the bound RE cut the parent nucleic acid). Maintaining the parent nucleic acid in this sufficiently stretched state enables the formation of the “train” of ordered fragments.

The digested fragments are released from the parent nucleic acid and continue to move downstream in the sheath fluid in the particular order in which they occurred in the parent nucleic acid from the leading end of the nucleic acid as it moves through a channel, for example, to the trailing end of the nucleic acid (which may be but need not be the order in which they were released from the parent). This will be described in greater detail herein.

The device is constructed and arranged to allow digested fragments to relax their conformations by reducing or eliminating the elongation force(s). Assuming a more relaxed form also causes gaps to form between digested fragments. Such gaps can be increased through further introduction of sheath fluid in the channel. In some instances, intercalator is introduced into the fluid stream at this point and is allowed to bind to the digested, relaxed fragments. (In other instances, the intercalator was combined with the parent nucleic acid prior to cutting, optionally prior to or at the same time as or after binding to RE.) Excess unbound intercalator may or may not be removed. The ordered digested fragments are then passed through an imaging system such as but not limited to a fluorescence microscope in order to measure the intensity such as fluorescence intensity of each relaxed fragment, which is proportional to the amount and thus length of the digested fragment.

The process can be repeated using a different RE, and the results can be used in combination to develop a signature or a more detailed map of the parent nucleic acid.

The ability to maintain the order of the digested fragments, as they exist in the parent nucleic acid from which they came, renders the device and approach superior to prior art methods including PFGE. The fragments are maintained in this order by performing the digestion while the parent nucleic acid (and correspondingly any released fragments) are completely stretched or nearly completely stretched. The ability to manipulate the parent and digested nucleic acids in flow allows for a wider range of manipulations to be performed and more finely controlled. The data so obtained can be used to replace or supplement sequence data obtained using for example PFGE. The device can be used to analyze simple as well as complex sample and nucleic acid mixtures. It will also yield quantitative information since it analyzes a single nucleic acid at a time as compared to a bulk analysis.

The methods provided herein may be referred to as non-hybridization methods since they do not require hybridization of nucleic acids to each other, such as for example hybridization of a parent nucleic acid to a nucleic acid probe, in order to obtain the nucleotide sequence of the parent nucleic acid. Rather the methods provided herein obtain the nucleotide sequence of the parent nucleic acid based on the cleavage of the nucleic acid by an RE of known sequence specificity. Additionally, the methods provided herein do not detect RE bound to the parent nucleic acids or their fragments. The REs therefore need not be and in some instances are not labeled with detectable labels, and nor must they be bound to the nucleic acid fragments while such fragments are being detected. Moreover, the nucleic acid fragments are detected based on signal from bound intercalator. This simplifies the detection system necessary to detect such fragments, since it must only detect signal from the intercalator, rather than signal from intercalator or fluorophore on another agent bound to the fragment, such as for example the RE or a hybridized probe, etc.

The methods provided herein may be performed on single nucleic acids such as single DNA molecules, and are thus referred to as “single molecule analysis” methods. The nucleic acids are typically not fixed or immobile (e.g., conjugated to a support such as a bead or a surface) and rather are in flow in a fluid stream.

Thus, one aspect of this disclosure provides a device for manipulating a polymer such as a nucleic acid (e.g., DNA) in a fluid sample, the device comprising a sample fluid inlet and a channel in fluid communication with the sample fluid inlet, the channel having a focusing region, a cutting region, a relaxation region, and a detection region. The focusing region may have a pair of supplementary inlets in fluid communication with the channel, the pair of supplementary inlets opposing one another on either side of the channel. The cutting region may have a converging width shape.

In some embodiments, the relaxation region has a first portion having a converging width shape and a second portion having a diverging width shape.

In some embodiments, the pair of supplementary inlets symmetrically oppose one another.

In some embodiments, at least a portion of the focusing region has a converging width shape.

In some embodiments, the converging width shape of the first portion of the relaxation region has a sharper rate of convergence than the converging width shape of the cutting region.

In some embodiments, the converging width shape of the focusing region is a different shape than the converging width shape of the cutting region.

In some embodiments, the converging width shape of the focusing region has a sharper rate of convergence than the converging width shape of the cutting region.

In some embodiments, the converging width shape of the first portion of the relaxation region is a different shape than the converging width shape of the focusing region.

In some embodiments, the supplementary inlets are connected to the channel at the focusing region of the channel.

In some embodiments, the device further comprises a second pair of supplementary inlets in fluid communication with the channel, the pair of supplementary inlets opposing one another on either side of the channel.

In some embodiments, the second pair of supplementary inlets symmetrically oppose one another.

In some embodiments, one of the supplementary inlets is positioned above the channel and the other supplementary inlet is positioned below the channel.

In some embodiments, a width of the converging width shape of the focusing region is proportional to a function of 1/x, where x is distance along the focusing region of the channel.

In some embodiments, the converging width shape of the cutting region has a linearly decreasing width.

In some embodiments, the converging width shape of the first portion of the relaxation region has a linearly decreasing width.

In some embodiments, the diverging shape of the second portion of the relaxation region has a linearly increasing width.

In some embodiments, the converging width shape of the cutting region has a constant strain rate profile.

In some embodiments, the converging width shape of the first portion of the relaxation region has a constant acceleration profile.

In some embodiments, the converging width shape of the first portion of the relaxation region may be a different shape than the converging width shape of the cutting region.

In some embodiments, the relaxation region has a straight section.

Another aspect of this disclosure provides a method for manipulating a nucleic acid in flow comprising: stretching a parent nucleic acid as the parent nucleic acid moves through a channel, digesting the nucleic acid, in flow, with a sequence-specific endonuclease to generate a plurality of digested fragments, and maintaining the digested fragments in a linear arrangement in flow that represents the order of the fragments in the parent nucleic acid.

In some embodiments, the method further comprises allowing the digested fragments to relax to cause gaps to form between the digested fragments and to cause the digested fragments to coil.

In some embodiments, stretching the parent nucleic acid comprises flowing the parent nucleic acid through a focusing region of the channel having a pair of supplementary inlets in fluid communication with the channel, the pair of supplementary inlets opposing one another on either side of the channel.

In some embodiments, the method further comprises introducing viscosifying agents into the channel through the pair of supplementary inlets.

In some embodiments, the pair of supplementary inlets symmetrically oppose one another.

In some embodiments, the focusing region has a converging width shape.

In some embodiments, allowing the digested fragments to relax comprises flowing the digested fragments through a relaxation region of the channel having a first portion with a converging width shape and a second portion with a diverging width shape.

In some embodiments, the nucleic acid is stretched by non-electrophoretic means.

In some embodiments, the nucleic acid is stretched and moved by hydrodynamic force.

In some embodiments, the digested fragments are run through a polymerase chain reaction.

Another aspect of this disclosure provides a method for manipulating a nucleic acid in flow comprising: digesting an elongated (or stretched) parent nucleic acid, in flow, with a sequence-specific endonuclease, to generate a plurality of digested fragments, maintaining the digested fragments in a linear arrangement in flow that represents the order of the fragments in the parent nucleic acid (from leading end to trailing end), and determining the length of each digested fragment. A device is described for implementing such a method.

In some instances, digestion may be but need not be sequential digestion. As used herein, sequential digestion means that the nucleic acid is digested in an ordered manner from one end (typically the leading end) to the other end (the trailing end), resulting in fragments in a linear order that mirrors the linear arrangement of such sequences in the parent nucleic acid prior to digestion. Digestion may occur randomly or at least in a different order and still the resulting fragments remain in an order that corresponds to the linear arrangement of such sequences in the parent nucleic acid prior to digestion.

In some embodiments, the sequence-specific endonuclease is a restriction enzyme. In some embodiments, the restriction enzyme is a type II restriction enzyme. In some embodiments, the restriction enzyme is a PD . . . D/ExK restriction enzyme.

In some embodiments, the elongated parent nucleic acid is in flow in a microfluidic channel. The microfluidic channel may have a diameter of about 10 microns or greater. In some embodiments, the method is performed in a microfluidic device.

In some embodiments, the length of each digested fragment is determined based on its signal intensity, such as fluorescence intensity. The digested fragments may be stained with intercalator prior to determining their length. In some embodiments, the digested fragments are stained with intercalator after digestion. In some embodiments, the digested fragments are stained with intercalator prior to digestion.

In some embodiments, the digested fragments are relaxed following digestion.

In some embodiments, the parent nucleic acid is elongated using hydrodynamic force. In some embodiments, the parent nucleic acid is elongated using electrophoresis such as gel-free electrophoresis. Electrophoresis can also be used to transport the nucleic acid from the binding condition to the cleaving condition. In some embodiments, a combination of hydrodynamic force and electrophoresis is used to transport the nucleic acids along the channel and/or through the device. In some embodiments, the parent nucleic acid and its fragments are moved using hydrodynamic force independent of electrophoresis (and thus may be characterized as non-electrophoretic).

Another aspect of this disclosure provides a method for obtaining sequence information from a nucleic acid comprising incubating a parent nucleic acid with a plurality of restriction enzymes (of identical type, for example, all are BamHI) under conditions that allow the REs to bind to but not cleave the nucleic acid, elongating the parent nucleic acid with bound REs while in flow, altering the conditions sufficiently to cause the bound REs to cleave the parent nucleic acid, thereby creating a plurality of digested fragments linearly arranged in flow, staining the digested fragments with an intercalator while maintaining the position of each relative to the other digested fragments, and measuring signal intensity, such as fluorescence intensity, of each digested fragment individually in a sequential manner, wherein the fluorescence intensity and detection order of the digested fragments, together with the sequence specificity of the RE yield a map of the parent nucleic acid. In some embodiments, the parent nucleic acid is stained with intercalator prior to, at the same time as, and/or after incubation with the plurality of REs but before digestion.

In some embodiments, the parent nucleic acid is a DNA. The parent nucleic acid may be on the order of 20-1000 kbp in length, or 20-750 kbp in length, or 20-500 kbp in length, or 50-500 kbp in length.

The digested fragments may be on the order of 500 bp to 100 kbp in length, or 1 to 50 kbp in length, or 1 to 20 kbp in length, or 1-10 kbp in length, or 1-7.5 kbp in length, or 1-5 kbp in length.

Another aspect of this disclosure provides a method for obtaining sequence information from a nucleic acid comprising incubating a parent nucleic acid with a plurality of restriction enzymes (of identical type, for example, all are BamHI) under conditions that allow the restriction enzymes to bind to but not cleave the nucleic acid, staining the parent nucleic acid having bound restriction enzymes with an intercalator, elongating the parent nucleic acid with bound REs while in flow, altering the conditions sufficiently to cause the bound REs to cleave the parent nucleic acid, thereby creating a plurality of digested fragments linearly arranged in flow, and measuring signal intensity, such as fluorescence intensity, of each digested fragment individually in a sequential manner, wherein the fluorescence intensity and detection order of the digested fragments, together with the sequence specificity of the RE yield a map of the parent nucleic acid. In some embodiments, the parent nucleic acid is stained with intercalator prior to, at the same time as, and/or after incubation with the plurality of REs but before digestion.

These and other aspects and embodiments will be described in greater detail herein.

### BRIEF DESCRIPTION OF DRAWINGS

Reference can be to the color version of FIGS. 1-19 in U.S. Provisional Application Ser. No. 62/431,398.

FIG. 1 is a schematic illustrating a method of analyzing nucleic acids according to one aspect. While the Figure refers to staining of DNA with PicoGreen®, it is to be understood that other intercalators can be used, including for example SYBER™ Green. Intercalators that do not significantly impact DNA structure upon binding to DNA may be used in such embodiments. Such intercalators are known in the art.

FIG. 2 is an embodiment of a device that may be used to implement the method of FIG. 1.

FIG. 3 is a streamline and heat map of the velocity field in the focusing region of the device. The “z” direction is along the chip depth.

FIG. 4 is a streamline and heat map of the velocity field in the cutting region of the device.

FIG. 5 is a vector and heat map of the velocity field in the relaxation region of the device.

FIG. 6 is a schematic of behavior of a nucleic acid in the flow acceleration-deceleration zones of the relaxation region of the device.

FIG. 7 is a streamline and heat map of the velocity field in a high aspect ratio channel.

FIG. 8 is a streamline and heat map of the velocity field in a relaxation region of a device having a plurality of parallel detection channels.

FIG. 9 depicts an optical setup for nucleic acid analysis.

FIG. 10 is a schematic of a plurality of individual parent nucleic acids bound to a plurality (more than one) RE of the same type. The Figure illustrates that the parent nucleic acids in the sample may not be identical, and may not be bound to the same number of REs. The totality of the parent nucleic acids may span the entire genomic DNA of a pathogen, or the entire genomic DNA of a plurality of pathogens. The method identifies the number and location of RE binding/cutting sites on a parent nucleic acid, thereby generating a “map” of those sites per parent nucleic acid. Those maps can then be overlaid to obtain a larger map for a given sample. In addition, the analysis can be performed with different REs (i.e., REs that bind/cut at different locations) sequentially, and the maps generated from each analysis can be overlaid as well for a more detailed genetic map.

FIG. 11 is a schematic of the plurality of parent nucleic acids each bound to REs flowing through a channel of narrowing cross-sectional area. The Figure illustrates that a channel with a converging width shape (which leads to decreasing cross-sectional area) can be used to apply an extensional strain on the nucleic acids, and this strain elongates the nucleic acids. The Figure also illustrates an embodiment in which the nucleic acids enter the narrowing channel individually. This can be achieved by reducing the concentration of the nucleic acids either prior to loading into the channel or during the movement of the nucleic acids through the channel. Importantly, the fragments are linearly organized and remain in the order in which they were present in the parent nucleic acid. This is achieved by maintaining the parent nucleic acid and the fragments in a completely stretched or nearly completely stretched state during the digestion step.

FIG. 12 is a schematic of a parent nucleic acid moving through a channel having side channels through which is flowed magnesium ions (Mg²⁺) or manganese ions (Mn²⁺), or a combination thereof. Following contact with these ions, the previously intact parent nucleic acid is cleaved into fragments (referred to herein as digested fragments or digested nucleic acids). The RE dissociates from the parent nucleic acid following cleavage. The digested fragments continue to move through the channel in an elongated state. Importantly, the fragments are linearly organized and remain in the order in which they were present in the parent nucleic acid.

FIG. 13 is a schematic of further passage of the digested fragments through the channel and the various modifications the nucleic acids undergo. First, the extensional strain is reduced with the result that the elongated fragments relax and adopt a more condensed or coiled state (illustrated as circles in the Figure). In some instances, the rate of relaxation can be further altered by including viscosity agents in the sheath fluid. The distance between the fragments can also be increased by introducing more sheath fluid into the channel via for example side channels, as illustrated. The distance between the fragments may be increased to ensure that the fragments are recognized and analyzed downstream as single events. Importantly, the fragments are linearly organized and remain in the order in which they were present in the parent nucleic acid.

FIG. 14 is a schematic of intercalation of the relaxed fragments as they flow through the channel. As discussed herein, staining with intercalator may occur prior to binding of RE, at the same time as binding of RE, after binding of RE, before cutting by RE, and/or after cutting by RE. The Figure provides one embodiment in which intercalator is introduced into the channel through a side channel. The Figure also illustrates a second set of side channels through which sheath fluid, including intercalator, is removed from the channel once intercalation occurs. The length of the channel between these two sets of side channels may vary depending on the time it takes to intercalate the nucleic acid sufficiently. Each nucleic acid will be intercalated to the same extent on average. Importantly, the fragments are linearly organized and remain in the order in which they were cleaved from their parent nucleic acid. Once intercalated and sufficiently separated from each other, each fragment is flowed through an excitation region which may comprise a laser (to excite the intercalator) and a detection region (to detect fluorescence emission from the intercalator). The excitation region and the detection region may be in the same area.

FIG. 15 is a schematic illustrating an embodiment according to one aspect. Cutting order refers to the order of the fragments as they existed in the parent nucleic acid.

FIG. 16 is a schematic illustrating an embodiment according to one aspect.

FIG. 17 is a photograph of a gel electrophoresis run of lambda DNA mixed with REs ApaI, SmaI and BamHI in binding only buffer. No cutting is observed.

FIG. 18 is a photograph of a gel electrophoresis run of lambda DNA mixed with REs ApaI, SmaI and BamHI in binding buffer (may be referred to herein as “binding only” buffer) spiked with cutting buffer as described in the Examples. The introduction of magnesium via the spiking process results in cutting of the DNA and produces expected digestion maps as shown.

FIG. 19 is a photograph of two tubes, the left tube comprising PicoGreen® in binding buffer and the right tube comprising DNA with PicoGreen® in the same binding buffer. Enhanced signal from the right tube is apparent.

FIG. 20 is an embodiment of a constant-width focusing region of a device.

FIG. 21 is an embodiment of a device according to one aspect.

FIG. 22 is a schematic illustrating an embodiment according to one aspect.

### DETAILED DESCRIPTION

Provided herein is a process for high-throughput analysis of nucleic acid samples that involves mapping nucleic acids based on number and relative location of RE binding/cleavage sites. Significantly, the methods provided herein maintain digested fragments in flow in the order in which they are present in a parent nucleic acid. These fragments are maintained in this order until they are analyzed by performing the digestion under conditions that maintain the nucleic acids, whether parent nucleic acids or fragments thereof, in a sufficiently elongated (or stretched) state. The ability to maintain this order provides further information about the relationship of the fragments relative to each other, and allows the parent nucleic acid to be reconstructed more efficiently and accurately. The ability to perform this method on a nucleic acid in flow allows for high-throughput capacity, among other things.

The method derives the following information about a parent nucleic acid: (1) the number of binding/cutting sites for the particular RE used (based on the number of fragments that are detected in a short window of time), (2) the distance between those binding/cutting sites (based on the fluorescence intensity of each detected fragment), and (3) the order of those binding/cutting sites (based on the order in which the fragments are detected).

The method is a significant improvement over prior art methods used to generate maps of genomic DNA. The method does not require fixation of the parent nucleic acid or of its digested fragments. The method analyzes single nucleic acids on an individual basis and therefore it does not require amplification of the original nucleic acid sample. It also does not rely on hybridization of probes as the indicators of sequence. Rather the sequence readouts are derived from the binding/cleavage sites of the REs used.

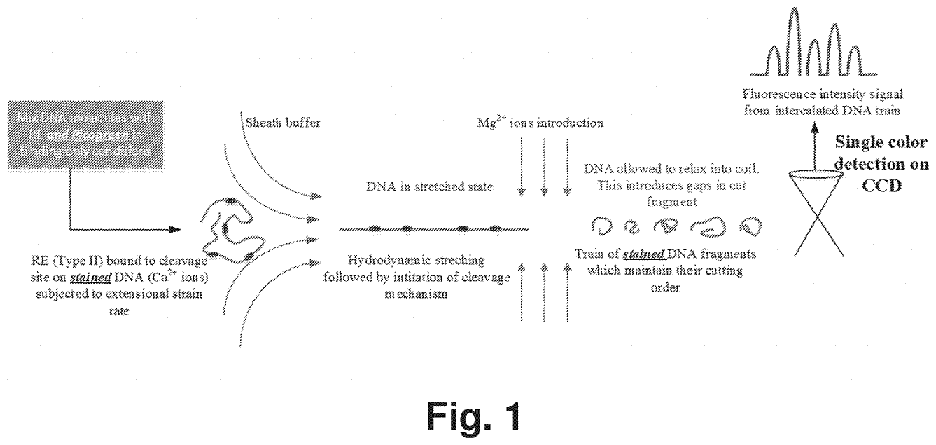

One embodiment of an approach for analyzing nucleic acids is provided in FIG. 1. This Figure illustrates the order of operations to be performed in some aspects of this disclosure for mapping a single nucleic acid (e.g., DNA) in a device of this disclosure. In some embodiments, the order of operation is DNA stretching, Mg2+ ion (or other suitable agent or condition) introduction, cutting of DNA (also referred to here as hydrolysis of DNA), gap formation between cut fragments, and optical detection.

It is to be understood that although various descriptions provided herein may refer to DNA these descriptions apply more broadly to nucleic acids in general unless explicitly stated otherwise.

In some embodiments, the device is designed to manipulate DNA. According to one aspect, using such a device, DNA need not be confined by device walls as is done in nanochannel based mapping platforms. In some embodiments, the device may be a microfluidic chip, and may comprise one or more nanochannels.

In some embodiments, the device may have a depth of at least about 0.5 μm, of at least about 1 μm, at least about 10 μm, at least about 100 μm or at least about 1 mm. In some embodiments, the device may have a depth of less than or equal to about 1 mm, less than or equal to about 100 μm, less than or equal to about 50 μm, less than or equal to about 10 μm, or less than or equal to about 5 μm. Combinations of the above-referenced ranges are also possible. For example, in some embodiments, the device may have a depth of about 0.5 μm to about 1 mm, or about 1 μm to about 100 μm, or about 1 μm to about 50 μm, or about 1 μm to about 10 μm. In some embodiments, the device may have a depth of 1 μm. In some embodiments, the device may have a depth of 100 μm.

In some embodiments, the use of Ca2+ ion in binding buffer may allow formation of a stable RE-DNA complex which in turn allows washing away of free (i.e., unbound) RE or makes the complex less sensitive to free RE concentration while manipulated in the device.

FIG. 1 illustrates the various steps starting from mixing the harvested DNA with RE, such as a type II RE, and an intercalator, such as PicoGreen®, in a binding condition (e.g., Ca²⁺, no Mg²⁺, no Mn²⁺), hydrodynamic stretching of the DNA and exposure of the DNA to the cleaving condition (e.g., introduction of Mg²⁺ ions), allowing the DNA to relax into a coiled state, optionally increasing the distance between digested fragments, detection of the fragments using for example a single color detector CCD, data collection and analysis. Subsequent steps such as creation of a map or signature of the parent DNA (based on the proportional relationship between fluorescence intensity and DNA length), pattern assembly/matching (e.g., arranging various fragments relating to each other, including with overlap between fragments), and optionally identification of one or more microbes in the original complex sample, are also contemplated. The DNA may be labeled with intercalator before and/or during and/or after incubation with RE, including optionally under the binding conditions, without significant (if any) impact on RE binding.

The mechanism of action of a RE can be split into several sub steps such as site searching, binding, hydrolysis, and product release. In some embodiments, the rate of the first step of RE binding to a specific site may be the only concentration-dependent process. In the presence of Ca2+ ions in binding buffer, the binding kinetics may become one-way towards formation of stable RE-DNA complexes. The cleavage or hydrolysis rate may be on the order of 10⁻¹ s⁻¹. In some cases, if DNA stretching can be accomplished at a rate faster than the RE cleavage rate, then stretching and Mg2+ ion introduction can be combined in one operation. With that in mind, in some embodiments, the fluidic chip may consist of three manipulation regions (a focusing region, a cutting region and a relaxation region) as discussed below.

As used herein, a nucleic acid in flow means a nucleic acid that is not attached at any point to a solid support. Thus, the nucleic acid moves along in a sheath fluid while the various manipulations and modifications described herein are performed.

### Device

The method may be performed in a device. In some embodiments, the device is a microfluidic device and may have a microfluidic channel having a sample inlet, a focusing region having a geometry that aids in stretching of nucleic acids flowing through the focusing region, a cutting region having geometry to keep the nucleic acid in a stretched state during digestion by RE, a relaxation region having a geometry that aids in relaxation of nucleic acids, and a detection region. In some embodiments, the device may have one or more supplementary inlets that allow the addition of agents or other fluids into the channel.

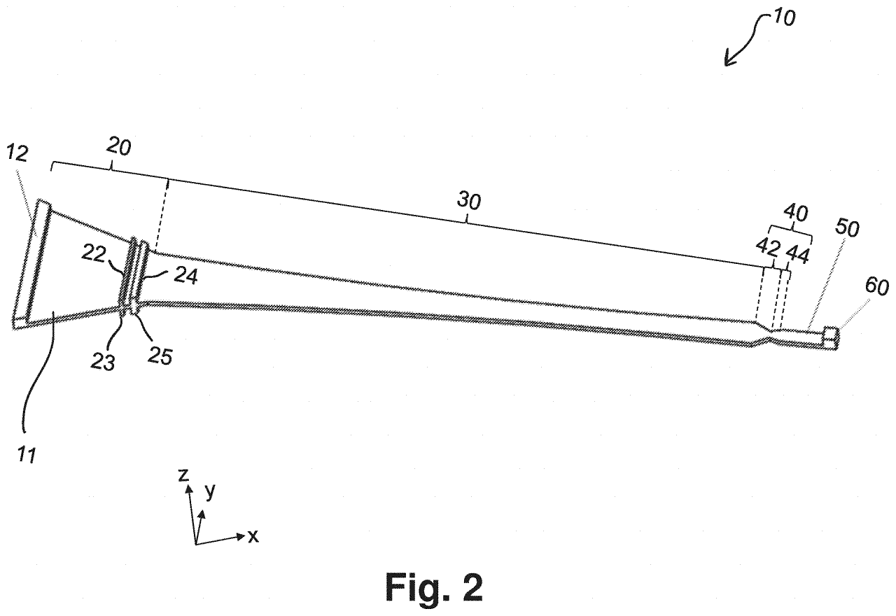

One illustrative embodiment of a device is provided in FIG. 2. The device 10 comprises a channel 11 through which a sample is flowed. The channel 11 has an inlet 12 for receiving the sample, and may include an outlet 60 through which sample is discharged. The channel may be a microfluidic channel. In some embodiments, the channel may have a plurality of regions, including: a focusing region 20, cutting region 30, relaxation region 40, and detection region 50. The features and functions of each region are discussed below.

### Focusing Region

According to one aspect, the channel of the device may include a focusing region that serves to stretch the nucleic acids within the sample.

In some embodiments, the focusing region may be used for one or a combination of the following functions: stretching the DNA, introducing cutting agents, introducing viscosifying agents, and/or reducing sample depth for mixing and focusing.

In some embodiments, the focusing region may include one or more pairs of supplementary inlets 22, 23, 24, 25 which may connect with the channel at the focusing region downstream of the sample inlet 12. These supplementary inlets may permit the introduction of sheathing fluid flows that help to stretch nucleic acids and/or position the sample layer at a suitable depth along the channel for detection. As used herein, the “depth” of the channel is the vertical dimension perpendicular to the direction of flow of the sample. In the coordinate axes labeled in the figures, the x-axis corresponds to length of the channel (and also coincides with the direction of flow of the sample), the y-axis corresponds to width of the channel, and the z-axis corresponds to depth of the channel.

In some embodiments, the supplementary inlets are arranged as a symmetrically opposing pair. In some embodiments, one of the inlets of the pair is positioned above the channel and the other inlet is positioned below the channel, such that the pair of supplementary inlets extend from the channel in the depth direction. As shown in FIG. 2, inlets 22, 23 form a symmetrically opposing pair in which inlet 22 is above the channel in the depth direction and inlet 23 is below the channel in the depth direction. In some embodiments, the device may have more than one pair of supplementary inlets. In FIG. 2, inlets 24, 25 form a second pair of symmetrically opposing inlets. While the supplementary inlets in FIG. 2 are shown vertically oriented such that they are perpendicular to the flow direction of the channel, it should be understood that the supplementary inlets may be angled instead. In some embodiments, where more than one pair of supplementary inlets are present, the size of each pair of inlets may be different. For example, in the embodiment shown in FIG. 2, the pair of inlets 22, 23 are smaller than the pair of inlets 24, 25.

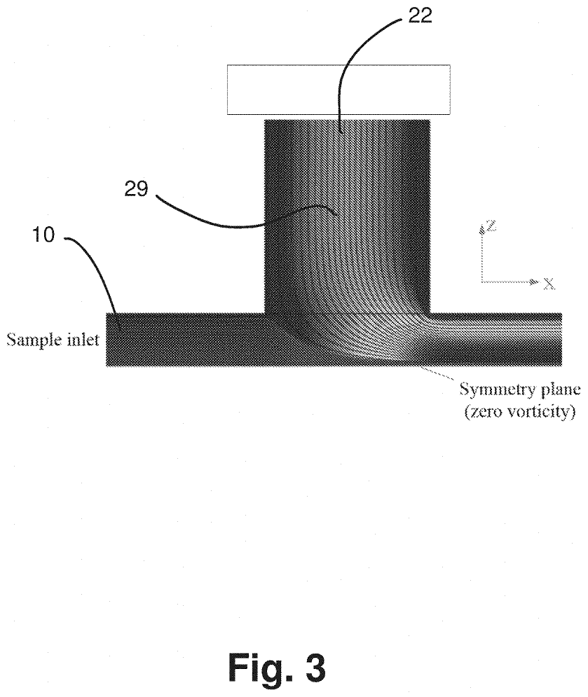

The supplementary inlets allow for the introduction of fluid streams that may help to focus the sample as it flows along the channel. FIG. 3 shows a flow field plot of the focusing region, demonstrating the effect of the introduction of a fluid stream 29 through a supplementary paired inlets 22, 23 on the sample layer (inlet 23 is shown in FIG. 2). In the flow field shown in FIG. 3 and all subsequent streamlines/flow fields shown in the figures, the spectrum of velocity goes from high to low in rainbow order (ROYGBIV, but without violet) such that red represents the highest velocity regions and dark blue represents the lowest velocity regions. In FIG. 3, with the channel midplane being the symmetry plane, only the top half of the channel is shown. The introduction of the fluid stream 29 focuses the sample along the depth direction z of the channel. Focusing the sample layer along the depth of the channel (along the z-axis) may increase the velocity of the sample and provide an elongation strain rate that causes the nucleic acids within the sample to stretch.

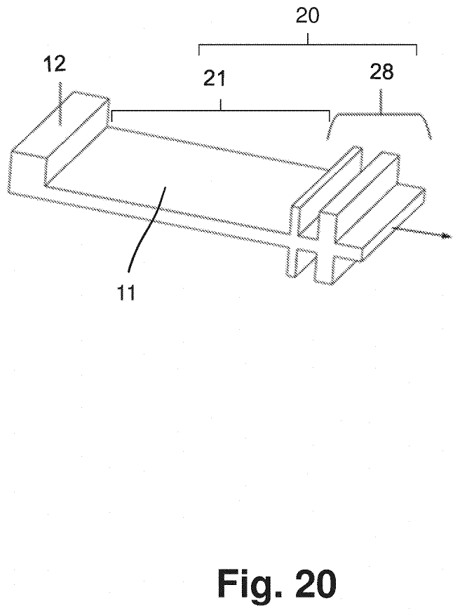

In some embodiments, the focusing region may have a converging width shape and/or may be preceded by a converging width shape section to aid nucleic acid stretching. In the embodiment shown in FIG. 2, the focusing region 20 has a converging width shape. In other embodiments, however, the focusing region may have a constant width shape rather than a converging width shape. One illustrative example of a constant width focusing region is shown in FIG. 20, which has a focusing region 20 having a pre-stretching zone 21 and a fluid flow focusing region 28 with supplementary inlets. In this embodiment, the channel 11 in the focusing region 20 has a constant width rather than a converging width.

In some embodiments, the focusing region may be used to expose the nucleic acids in the sample to a cleaving condition, e.g., introduction of Mg2+ ions or other agent or condition sufficient for cutting. In some embodiments, the supplementary inlets may be used to introduce the sample to such agents. Additionally, agents for increasing the relaxation time of nucleic acids can be introduced in this region. In some embodiments, such agents may be used to reduce the functional strain rates in the channel, which may lead to subsequent reduction in length of flow regions required in the channel. A channel could have a plurality of these regions for accomplishing the tasks described in FIG. 1.

In some embodiments, one or more viscosifying agents may be introduced to the sample through the supplementary inlets. Viscosifying agents will be discussed in greater detail in a later section.

In some embodiments, in the focusing region, the nucleic acids are stretched until RE sites are linearized. For example, in the case of lambda DNA being mapped with SmaI/BamHI enzyme, the DNA is stretched until fractional extension <x>/L˜0.85. This is based on the smallest DNA fragment length after cutting to the length of lambda DNA. In some cases, the Deborah number, defined as ET, (elongational strain multiplied by relaxation time) for achieving this is approximately 10. In some cases, stretching in shear may produce lower fractional extensions due to vorticity fields. Smith et al. [ref. 6] showed that stretching of DNA in lower De (˜1) may be beneficial for avoiding formation of slow stretching folded conformations. Larson et al. [ref. 7] demonstrated fast stretching of DNA in a microfluidic chip using hyberbolic contraction. Hyberbolic contraction may lead to linearly increasing extensional strain rate.

In some embodiments, one approach for generating increasing strain rate is to use focusing flow in the depth direction of the channel as shown in FIG. 3. In this case, the nucleic acids are pushed to a zero vorticity plane, which is the plane of symmetry that bisects the channel in the depth direction. The resulting final high strain rate stretching may be similar to nearly pure elongational flow.

When focusing is applied in the depth direction of the channel, it can be used to position the sample within the depth of field of an optical detection system. In some embodiments, the depth of field of an objective with a magnification of 20× and numerical aperture (NA) of 0.4 is approximately 5.8 microns while that of 40× and NA of 0.7 is around 1 micron (Nikon source).

According to one aspect, focusing flow allows the use of a channel with a larger depth (e.g., on the order of 100 microns), which may help to reduce clogging. In some embodiments, the channel depth is about 190 microns. Focusing in the depth direction may also help to substantially reduce the mixing time of Mg2+ ions and/or other agents (e.g., relaxation agents or other cleaving agents) with the sample by reducing the length scale for diffusion. The time scale for mixing is proportional to the square of the diffusion length scale. Thus, reducing the length scale for diffusion also reduces mixing time.

In some embodiments, the focusing region may perform DNA stretching in two stages.

In some embodiments, the Deborah number (De) is approximately 50 and the accumulated strain is greater than 3 (for example, for lambda DNA of size 48.502 Kbp). Such conditions may allow exiting nucleic acids to have a fractional extension of around 0.9.

In some embodiments, the transit time of nucleic acids may be less than the hydrolysis time to ensure that no cutting takes place while the nucleic acids undergo stretching. In some cases, the transit time is equal to or less than approximately 1 second.

In some embodiments, the sample depth at the channel exit is less than the depth of field of the detection system.

### Cutting Region

According to one aspect, the channel of the device may include a cutting region in which the nucleic acids are cut. In some embodiments, the region serves to keep the nucleic acids and their fragments stretched until the hydrolysis reaction by the REs bound to the nucleic acid is complete. According to some studies, the hydrolysis reaction rates for restriction enzymes such as BamHI, SmaI and EcoRV have been measured to be about 0.2 s-1 [refs. 1,8].

The cutting region may help to provide tension to keep the nucleic acids stretched. In some embodiments, the cutting region of the channel has a converging width shape to hydrodynamically provide tension on the nucleic acids. As shown in FIG. 2, the cutting region 30 of the channel has a converging width shape. In some embodiments, the converging width shape of the cutting region has a profile to produce constant extensional strain rate in the region. In some embodiments, the converging width shape of the cutting region has a linearly decreasing width. In some embodiments, the converging width shape of the cutting region has a non-linearly decreasing width. For example, in some embodiments, the width w of the converging width shape of the cutting region is proportional to a function of the inverse of the distance along the length x of the cutting region such that the width w is given by:

$$w ∝ fn ( \frac{1}{x} )$$

In some embodiments, the converging width shape of the cutting region is different than the converging width shape of the focusing region. In some embodiments, the width of the converging width shape of the cutting region decreases more steeply than that of the focusing region. In other embodiments, the width of the converging width shape of the cutting region decreases less steeply than that of the focusing region.

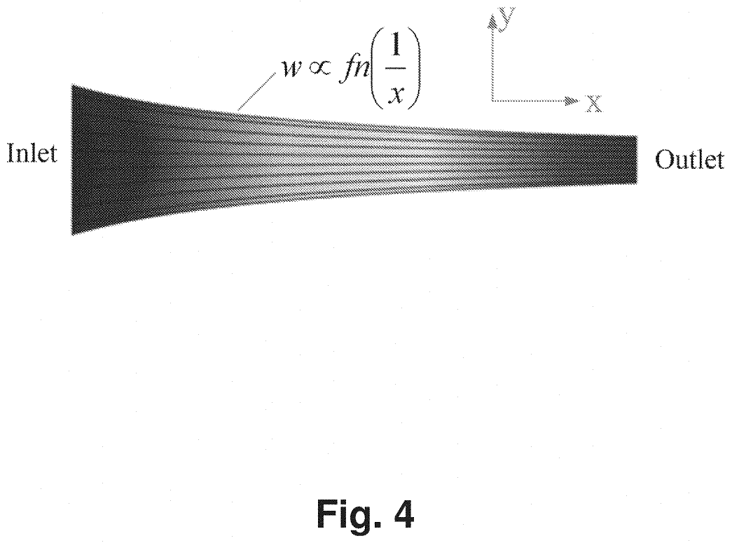

FIG. 4 is a streamline and heat map of the velocity field in the cutting region of the device. As seen in the Figure, velocity increases from the inlet of the cutting region to the outlet of the cutting region as the channel width of the cutting region decreases.

In some cases, nucleic acids, such as DNA fragments, will be in the stretched state if De ˜0.5. In the case of non-sequential digestion, the shortest fragment can be at the end of DNA molecule. Even in this worst-case scenario, the end RE site may be linearized if the condition of De ˜0.5 for a length 2 times the length of the shortest fragment expected after digestion is satisfied. For example, BamHI produces the shortest fragment of size 5.505 Kbp after digestion of lambda DNA. Thus, if the cutting region is designed to satisfy De ˜0.5 for a fragment of length 11.01 Kbp, then digestion may take place with DNA in a linearized state irrespective of the order of hydrolysis completion time of all five of the sites on lambda DNA.

In some embodiments, the transit time for molecules in the cutting region is at least about 1 second, at least about 2 seconds, at least about 3 seconds, at least about 4 seconds, at least about 5 seconds, at least about 10 seconds, at least about 15 seconds, at least about 20 seconds, or at least about 1 minute. In some embodiments, the transit time for molecules in the cutting region is less than or equal to about 1 minute, less than or equal to about 20 seconds, less than or equal to about 15 seconds, less than or equal to about 10 seconds, or less than or equal to about 5 seconds. Combinations of the above-referenced ranges are also possible. For example, in some embodiments, the transit time for molecules in the cutting region is about 1 second to about 1 minute, or about 2 seconds to about 20 seconds, or about 3 seconds to about 10 seconds. In some embodiments, the transit time for molecules in the cutting region is at least about 5 seconds.

In some embodiments, the cutting region may maintain the DNA in stretched state for the entire transit time of the molecules in the cutting region. In some embodiments, the cutting region may maintain the DNA in a stretched state for around 10 seconds. In some embodiments, the cutting region may maintain the DNA in a stretched state until hydrolysis is completed.

The transit time may help to ensure that the RE sites are linearized while cutting is completed by the restriction enzymes to preserve the order of the nucleic acid fragments. In some embodiments, the cutting region may not be long enough for a RE to cut more than one site i.e., the RE already bound to the nucleic acid is the only enzyme capable of completing the hydrolysis reaction on the nucleic acid during the transit time of the nucleic acid through the cutting region. This may ensure that any star activity in RE introduced by any of the agents input into the channel in the focusing region will not be consequential to the signature of the nucleic acid obtained at the end of the analysis process.

In some embodiments, the agents used to increase relaxation time that were introduced in the focusing region can help to reduce the strain rate requirement in the cutting region. This may help to keep the cutting region length short to satisfy the transit time requirement. FIG. 2 illustrates an example of a cutting region 30 with a focusing region 20 preceding it.

In some embodiments, the extensional strain rate in the region is such that the Deborah number is approximately 0.5 for a fragment length of 2 times the shortest fragment length expected after digestion.

In some embodiments, the transit time of nucleic acids such as DNA molecules is approximately 1.2 times the hydrolysis time to ensure complete digestion of RE-DNA sites only. In some cases, the transit time is approximately 6 seconds. The hydrolysis times for specific RE are known in the art.

### Relaxation Region

According to one aspect, the channel of the device may include a relaxation region that serves to introduce gaps in the digested nucleic acid fragments in order to help distinguish one fragment from another during detection.

In this region, the once-stretched nucleic acids are permitted to relax and consequently coil back. As a result, the nucleic acids are more compact, leading to improved detection signal.

In some embodiments, the relaxation region could have a straight width that extends for a long enough distance to allow nucleic acid to relax from a stretched state to a coiled state.

In other embodiments, the relaxation region is shaped to help accelerate the process of nucleic acid coiling and formation of gaps between digested nucleic acid fragments.

In some embodiments, the relaxation region may help to accelerate coil formation of digested fragments.

In some embodiments, to promote nucleic acid coiling, the relaxation region includes a flow acceleration zone followed by a deceleration zone. In some embodiments, the acceleration zone can be used to form gaps between stretched nucleic acid fragments, and the deceleration zone may create a deceleration flow field that aids in relaxation of the nucleic acids.

In some embodiments, the acceleration zone has a converging width shape and the deceleration zone has a diverging width shape. In some embodiments, the acceleration zone has a constant acceleration profile. In some embodiments, the acceleration zone has a width w that is proportional to a function of the inverse of the distance along the length x of the acceleration zone such that the width w is given by:

$$w ∝ fn ( \frac{1}{x} )$$

For example, the acceleration zone could have a linearly decreasing width, e.g., w=1/x. In other embodiments, the acceleration zone could have a non-linearly decreasing width, such as w=1/x² or w=1/x³.

In some embodiments, the deceleration zone has a width w that is proportional to a function of the distance along the length x of the deceleration zone such that the width w is given by:

w∝fn(x)

For example, the deceleration zone could have a linearly increasing width, e.g., w=x. In other embodiments, the deceleration zone could have a non-linearly increasing width, such as w=x² or w=x³.

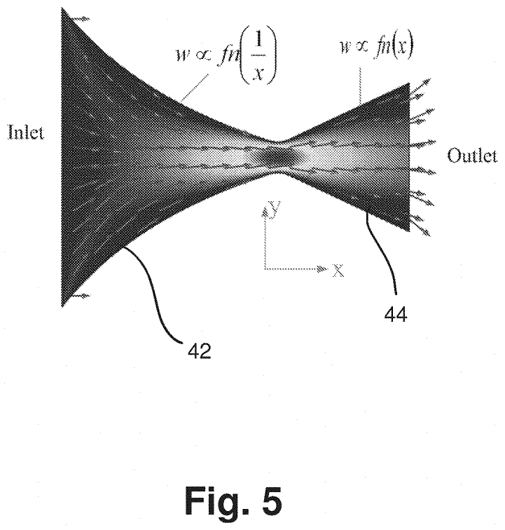

FIG. 5 depicts a schematic of a relaxation region having an acceleration zone 42 followed by a deceleration zone 44. The figure illustrates a vector and heat map of the velocity field in the relaxation region. FIG. 5 shows that the area of highest velocity is at the region of smallest width, and the areas of lowest velocity are at the regions of greatest width and toward the walls of the channel.

Without wishing to be bound by theory, in some cases, relaxation time is directly proportional to the coefficient of friction between nucleic acids and surrounding medium. In the case of Lamda DNA digested with BamHI, the longest fragment expected is 16.841 Kbp. The longest relaxation time expected for 16.841 kbp fragment in fluid with viscosity of 1 centipoise is approximately around 13 milliseconds. If the frictional coefficient is increased 100 times through addition of agents in the focusing region, then the relaxation time would be on the order of 1 second.

In some embodiments, the total accumulated strain in the deceleration zone is equal to or greater than that necessary to coil, fully or partially, the longest digested fragment from its stretched state. In the deceleration zone, the gap between fragments may reduce due to compression of fluid material elements. The acceleration zone which precedes the deceleration zone may increase the gap so that, when the fragments coil, the desired gap is attained. In some cases, the extensional strain rate applied does not produce tension above 65 pN in stretched nucleic acid (e.g., DNA) fragments.

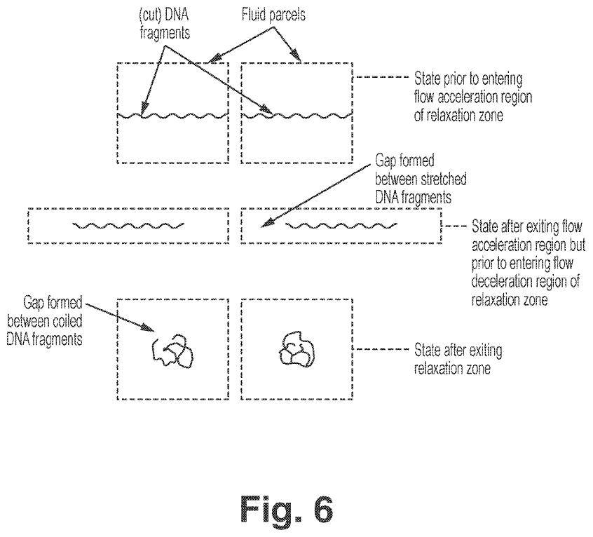

FIG. 2 depicts an illustrative embodiment of a relaxation region 40, which includes an acceleration zone 42 followed by a deceleration zone 44. Here, the acceleration zone 42 is a constant acceleration zone 42 created by converging walls. FIG. 6 explains the relaxation concept through an illustration of digested nucleic fragments and fluid parcel (material element) around the fragments. The flow acceleration is used to create gaps large enough such that, as the nucleic acid fragments flow through the deceleration region and begin to coil, gaps between coiled nucleic fragments remain.

In some embodiments, the accumulated strain in the deceleration zone is given by:

$${ɛ}_{d} = ln ( \frac{{x}_{f}}{{x}_{i}} )$$

where ‘xᵢ’ is the fragment length of the longest digested nucleic acid (e.g., DNA) fragment and ‘x_{f}’ is the desired diameter of the coiled fragment, e.g., 1 micron.

In some embodiments, the accumulated strain in the acceleration zone is given by:

$${ɛ}_{a} = ln ( \frac{{x}_{f}}{{x}_{i}} ) + {ɛ}_{d}$$

where ‘xᵢ’ is the fragment length of the longest digested nucleic acid (e.g., DNA) fragment with one site uncut and ‘x_{f}’ is the ‘xᵢ’ length plus the gap distance desired between coils, e.g., say 5 microns.

In some cases, the tension imparted to digested nucleic fragments should not exceed 65 pN. Tension can be adjusted by controlling the strain rate.

### Detection Region

According to one aspect, the device includes a detection region in which the nucleic acid fragments are detected and identified. In some embodiments, the detection region may be part of the channel. In some embodiments, the channel walls of the detection region may be straight.

As shown in the illustrative embodiment of FIG. 2, the device includes a detection region 50 with straight channel walls.

It should be understood that a variety of detection arrangements may be used to detect the nucleic acid fragments. In some embodiments, fluorescent intensity from the nucleic acid fragments is collected by an optical setup.

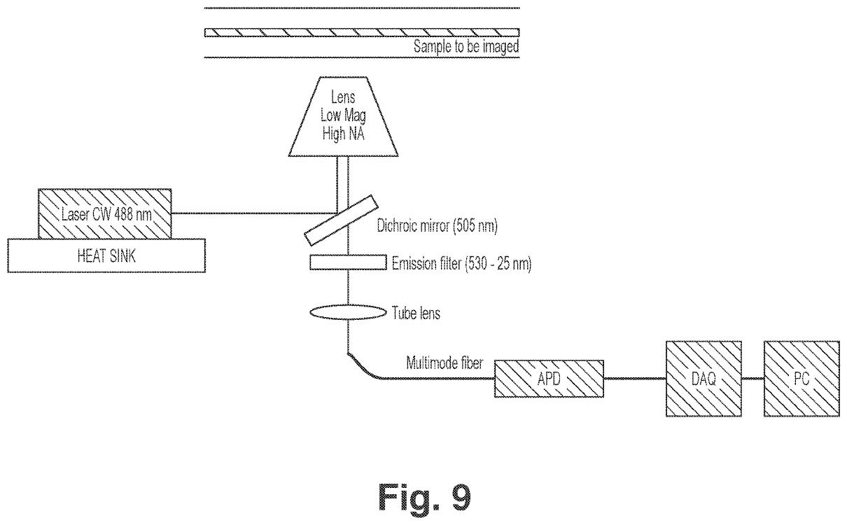

One illustrative embodiment of a detection arrangement is shown in FIG. 9, in which an optical setup is used to capture fluorescent intensity from PicoGreen®-stained nucleic acids (e.g., DNA). The dye is excited by 488 nm laser light and the fluorescent light emitted is recorded by a photodetector or an array of photodetectors.

In some embodiments, a high aspect ratio channel (e.g. width to depth ratio) may produce a velocity field which is independent of location along the width of the channel except close to corners. In some embodiments, an aspect ratio of greater than 5 can be considered a high aspect ratio channel. In one illustrative example, a channel having a depth of 100 microns and a width greater than 0.5 mm would be considered high aspect ratio channel.

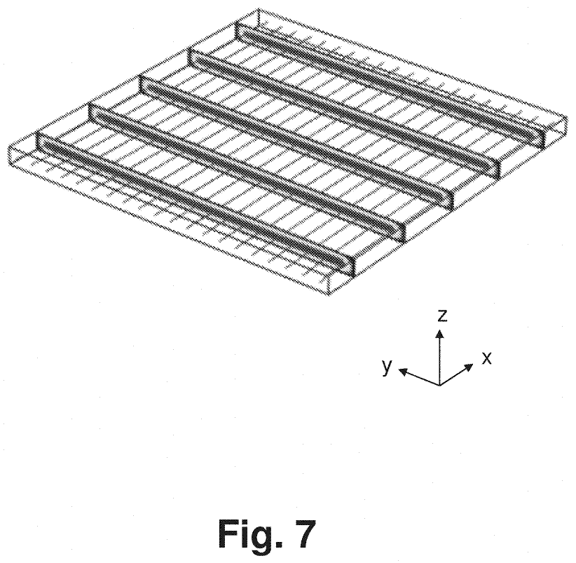

In some cases, high aspect ratio channels may help to ensure that nucleic acids in the sample that are separated along the width of the channel experience similar flow fields. FIG. 7 depicts a streamline and heat map of a velocity field of a high aspect ratio channel having width w and depth d. As seen in the Figure, the velocity field is generally uniform along the width of the channel, with lower velocity at the side walls and at the top and bottom surfaces of the channel. In some embodiments, the channel of the device has a high aspect ratio in one or more regions of the channel, e.g., the focusing region, cutting region, relaxation region, and/or the detection region.

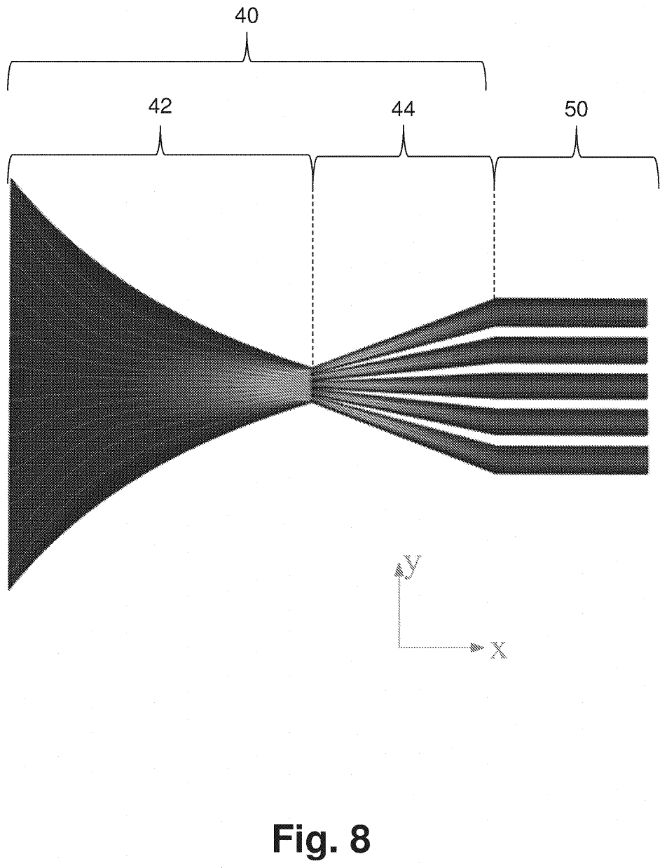

In some embodiments, the channel of the device may split into a plurality of channels. Such an arrangement may be used where multiple detectors are used. Spaced apart channels may help to accommodate the physical size of the detectors and the spacing between detectors. In some embodiments, the split into the plurality of channels may occur at the relaxation region. In some embodiments, the split occurs at the start of the deceleration zone. For example, in the embodiment shown in FIG. 8, the channel of the device splits into five separate channels at the deceleration zone 44 of the relaxation region 40, resulting in five separate detection channels in the detection region 50. It should be appreciated that the channel may be split into any suitable number of channels.

### Illustrative Embodiment

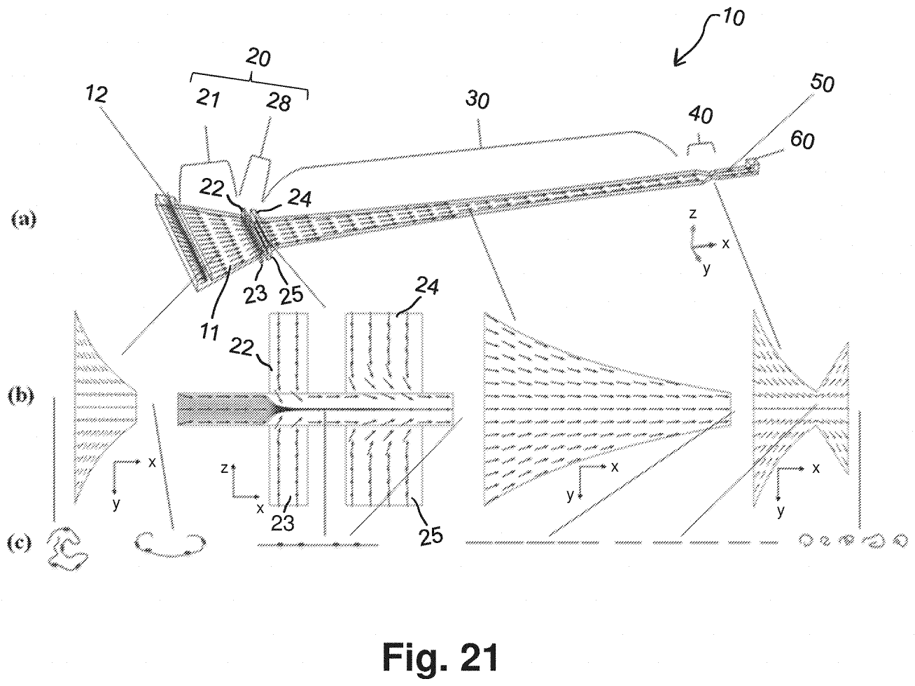

One illustrative embodiment of a device is provided in FIG. 21. Row (a) of the figure depicts a perspective view of the device with associated velocity vector fields represented by arrows. Row (b) of the figure shows planar views of the device at various locations of the device. The lines connecting the device of row (a) to the planar views of row (b) indicate the location of each planar view along the device. Row (c) of the figure represents the state of a stained DNA strand at various points along the device as it travels through the device. The embodiment of FIG. 21 includes a fluid flow focusing region 28, which includes supplementary inlets 22, 23, 24 and 25. It should be appreciated that, in the fluid flow focusing region 28, the width of the channel 11 may be converging or may have a constant width. The device may include a pre-stretching zone 21 which may have a converging or a constant width.

In some embodiments, the pre-stretching zone 21 may have a Deborah number of about 0.8. The transit time for a DNA molecule in the pre-stretching region may be, in some embodiments, at least on the order of the longest relaxation time of the DNA molecule. In some embodiments, the transit time is about 2 tau, where tau is the longest relaxation time of the DNA molecule.

In the fluid flow focusing region 28, in some embodiments, the tension on the DNA does not exceed 65 pN. In some embodiments, the transit time through the fluid flow focusing region 28 is less than 1 second.

In the cutting zone 30, in some embodiments, the conditions enable the smallest fragments, e.g. 5 Kbp long fragments, to be stretched for a long enough time to complete digestion. In some embodiments, the transit time through the cutting zone is about 10 seconds.

In the relaxation zone 40, in some embodiments, the tension on the DNA does not exceed 65 pN. In some embodiments, the overall accumulated strain in the relaxation zone is greater than zero.

### Viscosifying Agents

As discussed previously, certain agents may be introduced into the device channel to increase the relaxation time of the nucleic acids in the sample. This would reduce the fluid strain rate requirement which in turn allows completion of cutting in a shortened cutting region length. In other words, by using such agents it is possible to vary the length of the cutting region including to shorten the length of the cutting region while still achieving complete RE specific cutting of the nucleic acid. These agents are regarded as viscosifying agents. The viscosifying agents may reduce the fluid strain rate requirement to keep nucleic acids stretched, which may in turn lead to a reduction in fluid velocities and hence may permit a reduction in cutting zone length.

In some cases, addition of these viscosifying agents may lead to an increase in osmotic pressure. The increase in osmotic stress that is due to the addition of the agent however may increase the likelihood of cleavage at non-cognate sites (referred to here as star sites) which differ from cognate (correct) sites by, for example, a single base pair [ref. 9]. Site specificity of RE however can be restored by increasing the mechanical pressure in the cutting region [ref. 2]. Another approach for reducing such star activity (i.e., RE cutting at non-cognate sites) involves introducing a wash step to remove unbound RE from solution just after the binding protocol (or step) is completed (e.g., in a test tube). Unbound RE may be removed by any known method including dialysis and filtration. Once the unbound RE are removed, the only REs in the cutting region are the ones which are bound to their cognate sites. The probability of any such bound RE cutting the site to which it is bound, then subsequently finding, binding to, and cutting a star site within the 5 to 10 second transit time through the cutting region, is lowered.

Notwithstanding the foregoing, in some cases, star site hydrolysis can nevertheless be used to generate a unique map of DNA, and it can be promoted through addition of these agents even, for example, in binding buffer.

Another way of increasing the nucleic acid relaxation time without significantly impacting the osmotic pressure is to use mid to high molecular weight (MW) agents as viscosifiers. As provided herein, high MW agents can be used as viscosifiers even at low molar concentrations. Such low molar concentrations do not significantly impact osmotic pressure, which is typically proportional to molar concentration. Thus, one can use a high MW agent at low molar concentration and still effect a sufficient level of viscosity without significant impact on osmotic pressure. It should therefore also not lead to an increase in star activity.

Accordingly, an increase in fluid viscosity can be accomplished using low or mid or high MW agents to increase the friction coefficient, which in turn increases the relaxation time of a nucleic acid such as DNA. Mid or high molecular weight agents may also function as macromolecular crowding agents that can increase the reaction rates of enzymes, such as the REs of this disclosure. Low and mid or high MW agents are discussed in greater detail below. It is to be understood however that other agents may also be used to effect these same results.

Low MW Agents

Increasing the friction coefficient of nucleic acids such as DNA can be achieved using for example low MW agents. Examples of low MW viscosifying agents include glycerol (MW=92.094 Da) and sucrose (MW=342.30 Da). As used herein, a low MW agent is one having a MW substantially smaller than that of the RE being used. In some instances, the molar mass is equal to or less than 1000 Da, as a typical RE has a molar mass on the order of about 10 kDa. Thus, the agent may have a MW that is equal to or less than 10%, 5%, 1%, 0.1%, 0.01%, 0.001% or less than the MW of the RE that is being used.

As an example, an aqueous glycerol solution that is 88% by volume and 90% by weight, 12.04 M, at a temperature of 22° C., increases the dynamic viscosity of a fluid to approximately 200 cps [ref. 16]. This results in an increase in the friction coefficient and concomitant DNA relaxation time by about 200-fold as compared to those parameters in water. This increase leads to a proportional reduction in the strain rate required to keep the DNA molecule stretched while hydrolysis of all sites is completed. A lower strain rate also helps to reduce the final velocity of a DNA molecule and hence reduces the length of the cutting region along the flow direction.

The increase in viscosity may be associated with an increase in osmolality of the solution in the cutting region. As an example, the osmotic pressure, pi, of a 12.04 M glycerol solution is 291.75 atm (pi=cRT, where c is the molar concentration of agent, R is the universal gas constant, and T is the temperature of the fluid). High osmotic pressure has been shown to induce star activity [refs. 2,3]. However, if the transit time of DNA in a high osmolality condition in the cutting region is kept short (e.g., less than 10 seconds), there may not be sufficient time for an RE to bind and cut a star site, especially if the unbound (free) REs are washed away before loading the sample in the device. In some embodiments, the mechanical pressure in the device may be increased to reverse the effects of the increased osmotic pressure.

Mid or High MW Agents

Increasing the friction coefficient of nucleic acids such as DNA can also be achieved using for example mid or high MW agents such as Triton X-100, Tween 20, Poloxymer 407 and Dextran. As used herein, a “mid MW agent” is one having a MW in the range of greater than 1 kDa to about 100 kDa. Such agents may have a MW that is about 5 kDa to about 100 kDa, or about 5 kDa to about 50 kDa, or about 5 kDa to about 20 kDa, or about 5 kDa to about 10 kDa, or about 5 kDa to about 500 kDa, or about 5 kDa to about 200 kDa, or about 10 kDa to about 100 kDa, although they are not so limited. Some of these agents (e.g., Triton X-100) can even self-assemble and form even larger complexes when dissolved in aqueous solution, and thus the disclosure contemplates use of such agents at concentrations and/or under conditions that cause such macromolecular complexes to form.

Non-ionic detergents like Triton X-100 and Tween 20 are typically used in restriction enzyme buffers to prevent aggregation. A variety of restriction enzymes have been shown to work even at concentrations as high as 30% Triton X-100 without compromising their specificity [ref. 17]. The viscosity of 30% Triton X-100 at 25° C. is approximately 80 cps.

High MW agents can also be used without impacting overall restriction activity on DNA [ref. 4]. Molecular crowding can provide a soft fluid environment similar to DNA in nanochannels where the friction of DNA with molecular crowders increases its relaxation time significantly [ref. 10]. As used herein, a “high MW agent” is one having a MW of greater than 100 kDa. Its MW can range up to 200 kDa, 300 kDa, 400 kDa, 500 kDa, or more.

Other viscosifying agents (also referred to as vicosifiers in the art) include but are not limited to polyethyleneglycol (PEG), PVP, amphiphilic polymers such as hydropxypropyl cellulose, liquid paraffin, polyethylene, fatty oils, colloidal silica or aluminum, propylene glycol, propylene carbonate, carboxyvinyl polymers, magnesium-aluminum silicates, hydrophilic polymers (such as, for example, starch or cellulose derivatives), water-swellable hydrocolloids, carragenans, hyaluronates, alginates, and acrylates.

### Method

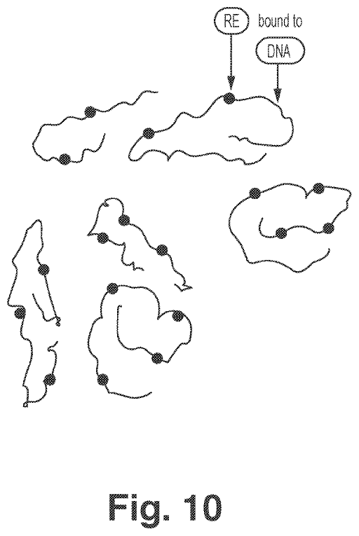

The steps of an embodiment of a preparation and analysis method according to some aspects are described in greater detail now. Reference can be made to FIGS. 10-14.

As a first step, a single or a plurality of parent nucleic acids such as parent DNA are mixed with a plurality of REs. The REs may be virtually any RE including those classified as PD . . . D/ExK RE. Significantly, the mixing of the RE and the parent nucleic acid occurs under conditions that promote binding of the RE to the parent nucleic acid but are not conducive to cleavage of the nucleic acid by the RE. This can be accomplished by using calcium ions in the solution instead of magnesium or manganese ions. The mixture is incubated until RE binding to all available specific sites is accomplished. A schematic of parent nucleic acids bound to the REs is provided in FIG. 10. This step may also involve mixing the parent nucleic acids with intercalator, before, during and/or after mixing with the RE.

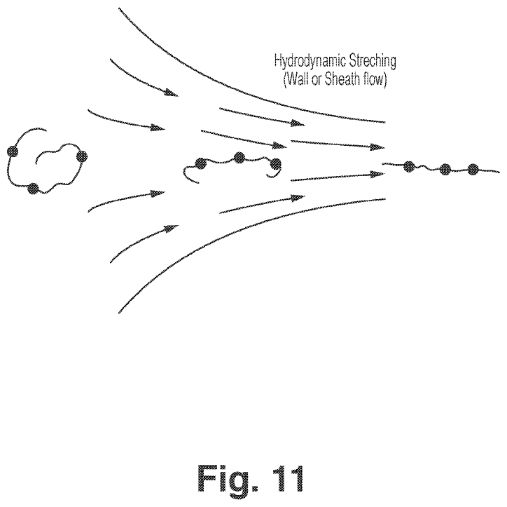

As a second step, the parent nucleic acid is hydro-dynamically stretched. This can be accomplished by modulating the fluid strain rate also known as extensional strain rate. In one embodiment, the fluid strain rate, epsilon, is modulated such that it is nearly equal to or greater than ‘1/tau’ where tau is the longest relaxation time of the parent fragment. The extensional strain rate can be modulated using channel wall geometry or sheath flow velocity (or flow rate) or both. The disclosure contemplates that this and subsequent steps are performed in a micro-fluidic device. FIG. 11 illustrates an embodiment in which the channel wall geometry creates a constriction for flow which in turn increases the fluid velocity and creates an extensional strain rate in flow field. Hydrodynamic focusing and extension of nucleic acids has been described by Wong et al. J. Fluid Mech., 497:55-65, 2003. A completely stretched nucleic acid has an end to end distance that is greater than 0.8 times its contour length. In some instances, the end to end distance of the parent nucleic acid may be less than 0.8 times its contour length, provided such nucleic acid is still sufficiently stretched to render the RE binding and cleavage sites linearized in space.

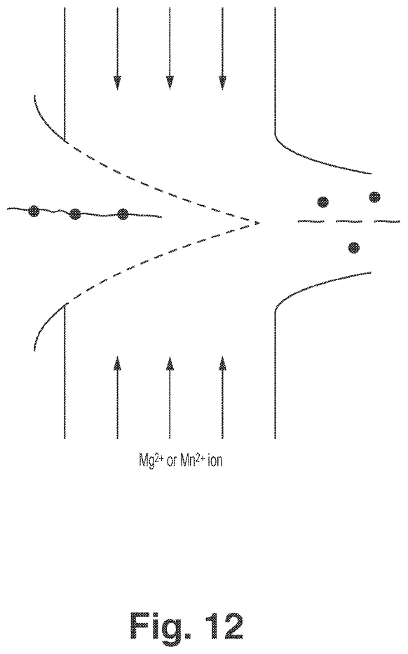

As a third step, when the parent nucleic acid is at or near a fully stretched state, Mg²⁺ or Mn²⁺ ions are introduced. The introduction of these ions will initiate digestion of the parent nucleic acid. These ions can be introduced into the flow stream for example through a porous wall in the channel and/or by using sheath fluid comprising one or both ions. FIG. 12 illustrates sheath fluid with either Mg²⁺ or Mn²⁺ ions introduced in a manner that causes digestion of the stretched parent nucleic acid to occur in an ordered manner. It is to be understood that digestion may occur in an ordered manner or it may occur in a simultaneous manner or it may occur in a random manner. Digestion is likely to occur so quickly and may therefore appear to be simultaneous or nearly simultaneous. Regardless, the order in which the bound RE cut their respective sites is less important than maintaining the nucleic acids at or near fully stretched states. The fragments will be on the order of 1 kbp (kilo base pairs) or longer. It will be understood that the fragments will be of different lengths, since their lengths will depend on the location of the RE binding/cleavage sites.

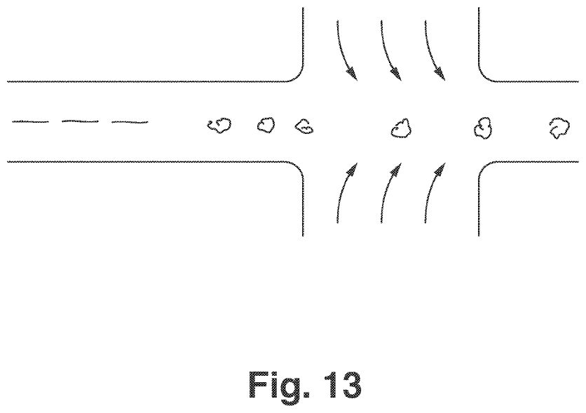

As a fourth step, once digestion has occurred, the digested fragments are now allowed to relax from their stretched state to a more relaxed state by either eliminating the extensional strain rate, epsilon, or by maintaining epsilon at a value that is less than that required to stretch DNA fragments. This process will increase the distance between the digested DNA fragments, thereby creating gaps. These gaps can be increased by introducing sheath fluid as shown in FIG. 13. One or more viscosity agents may be introduced here as well in order to reduce the relaxation rate.

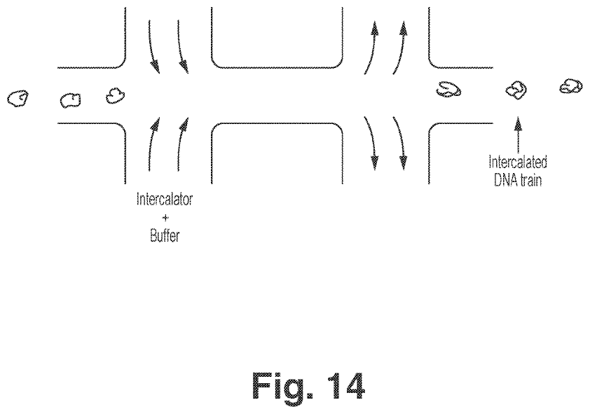

Optionally, as a fifth step, the ordered series (or train) of digested fragments can be intercalated. A pure diffusion process can be used to exchange sample fluid (fluid carrying the nucleic acid fragments) in the channel with a sheath fluid containing an intercalator. This too can be accomplished using channels with a porous wall or by introducing intercalator as shown in FIG. 14.

As a sixth step, the train of intercalated digested and relaxed fragments is passed through an imaging system such as a fluorescence microscope which yields a fluorescence intensity measurement for each fragment that is proportional to length of the digested fragment (Huang et al. Nucleic Acids Research, 24(21):4202-4209, 1996). The calculated length of the digested fragment can then be used to estimate the cleavage site of the RE on the parent nucleic acid.

The process can be repeated one or more times to find cleavage sites on another parent nucleic acid, which may be identical to or different from the first analyzed parent nucleic acid. Spacing between parent nucleic acids can be controlled by adjusting the concentration of such fragments when mixed with RE in step 1. The spacing between parent nucleic acids is kept larger than spacing between digested fragments.

The channels and detection regions can be arranged adjacent to each other and processing and detection through each of the channels can occur simultaneously. In this way, the same sample or multiple samples may be processed concurrently. If the same sample is present in a number of channels, then each channel may comprise nucleic acids bound to a different RE.

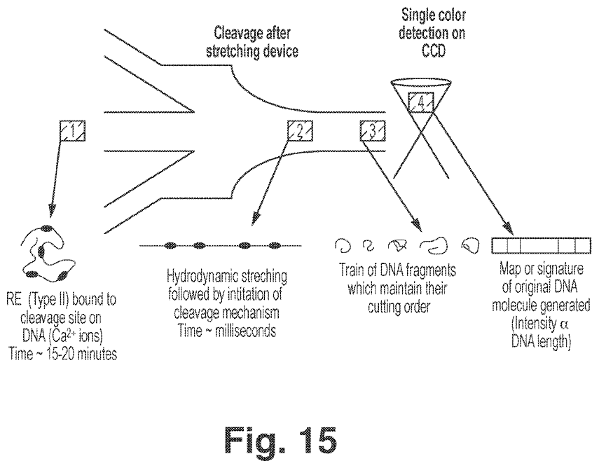

A combination of the foregoing steps is illustrated in FIG. 15, which depicts an alternative embodiment of the device of FIG. 2. The device in FIG. 2 includes (1) an inlet, optionally with an adjacent reservoir, (2) a first microfluidic channel downstream of the inlet and optional reservoir, the channel having (i) a first region having a geometry that imposes upon polymers moving therethrough an extensional strain rate. In some embodiments, the extensional strain rate is about equal to or greater than 1/tau where tau is the longest relaxation time of the polymers (referred to as an elongation region). The channel may also have (ii) a second region having parallel walls and that does not impose an extensional strain rate on a polymer moving therethrough (referred to as a relaxation region), (3) a first, second and third set of side channels located along the length of the second region of the microfluidic channel, and (4) a detection region within the second region. In another embodiment, the device may comprise electrodes at either end in order for electrophoretic movement of negatively changed DNA to occur through the device and channel(s).

In FIG. 15, a type II RE is bound to its cleavage site on the parent nucleic acid in the presence of Ca²⁺ cations (denoted as 1). This binding step may be performed in a microfluidic device (e.g., in a reservoir of such device) or it may be performed separately from the device and the RE-bound nucleic acids can then be loaded into the device. The binding of RE to the nucleic acids may take on the order of about 15-20 minutes, although it is possible that such incubation may be shorter or longer depending on the temperature of the solution, the particular RE used, etc. Such parameters are known to those of ordinary skill in the art. The solution may be referred to as a binding buffer, and it may contain Ca²⁺ cations as discussed herein, as well as buffering agents such as but not limited to Tris, EDTA, and NaCl. It will not contain Mg²⁺ and Mn²⁺ at all or it will not contain these ions at single or combined concentrations that facilitate cleavage of the nucleic acids by the bound RE.

Once the RE-bound nucleic acids are loaded into the microfluidic device, they are hydrodynamically stretched (denoted as 2). The stretching may be accomplished in a number of ways. In the Figure, stretching is accomplished using a two side streams that merge with a middle stream in a microchannel. In this configuration, the flow accelerates in the direction of movement (left to right). The stretching step can take on the order of milliseconds, and may be performed through a change in geometry of the microfluidic channel through which the nucleic acids are travelling. Movement of the nucleic acid as well as stretching of the nucleic acid may be performed using electrophoresis, instead of or in addition to hydrodynamic force. Microfluidic channel diameters may be, on average, greater than 10 microns. The microfluidic device is relatively simple in that it does not contain a post array in order to separate or elongate the nucleic acids. It also does not contain a semi-solid or solid matrix such as a gel. And it does not require any particular surface chemistry. For example, the nucleic acids simply move through the channels and are not attached to the channel walls and accordingly the interior surface of those walls do not comprise any chemical substituents required for binding of nucleic acids.

Once the nucleic acids are stretched, each is individually cut a plurality of times (the plurality dependent on the number of bound RE). Such cutting may start from the leading end of the nucleic acid and finish at the trailing end of the nucleic acid, in some non-limiting embodiments. Alternatively, cutting may occur simultaneously or randomly. The disclosure contemplates all of these possibilities. This can be accomplished by exposing the nucleic acid to a cleaving condition, such as for example Mg²⁺ and/or Mn²⁺ at a single or combined concentration that facilitates cleavage by the bound RE. As the nucleic acid moves through the region of the channel comprising the cleaving condition, it gets cleaved, and its released fragments are maintained in an ordered linear arrangement, with each fragment cleaved from the parent nucleic acid continuing to move downstream in the channel.

Following cleavage, the RE dissociates from the nucleic acid. In addition, the released nucleic acid fragments are no longer elongated (due to a change in channel geometry or a change in sheath fluid dynamics) and are able to assume their native coiled state (denoted as 3). In some instances, a viscosity agent may be used (or may be introduced) to slow the relaxation rate of the nucleic acid fragments. Thus, it is contemplated that the sheath fluid comprises a viscosity agent throughout the process or that a viscosity agent is introduced into the sheath fluid after cutting of the nucleic acid. In the presence of the viscosity agent, the nucleic acid fragments adopt their native coiled state at a slower rate (as compared to in the absence of the viscosity agent), which in some instances is desirable. An example of such a viscosity agent is glycerol. Other viscosity agents are known in the art, including without limitation sucrose, and polymers such as polyethylene glycol (PEG), polyacrylamide or polyvinyl alcohol (PVA) all of which may be provided in aqueous solution forms.

In some instances, the released nucleic acid fragments may be exposed to intercalator following cleavage as they travel downstream through the channel. Once sufficiently intercalated, the nucleic acids are detected in the order in which they are travelling. In other embodiments, the parent nucleic acid may be exposed to intercalator before binding to RE, while binding to RE, and/or after binding to RE, including before cutting with RE.

Detection intends that the signal such as fluorescent signal from each fragment is detected and measured. The amount of fluorescence emitted from each fragment is proportional to the amount of intercalator bound to the fragment which is itself proportional to the length of the fragment. Thus, by measuring the amount of fluorescent signal from each fragment, it is possible to determine the length of such fragment, and thus the distance between two consecutive binding/cleavage sites for the RE that was bound to the nucleic acid. Only a single color (or wavelength) detection system is required because only fluorescence from the intercalator is being detected. Moreover, fragment detection is based on detection of the totality of intercalators bound to each fragment. Since each fragment will be bound to hundreds or thousands of intercalators, the signal from each fragment will be sufficiently strong to be detected. The signal to noise ratio will be much higher than methods that rely on hybridization and detection of signals from single probes. The analysis of a plurality of nucleic acids simultaneously can be achieved using parallel channels and detection regions, as may be accomplished with for example linear detector arrays such as CCD arrays. The process is able to process nucleic acids from greater than 10⁷ cells in about 30 minutes.

It will be apparent that no hybridization occurs and that the method does not ultimately detect the bound RE. It will also be apparent that the method does not involve fixation of nucleic acids, and instead relies on the parent nucleic acids and their digested fragments to move in flow or in solution along a channel such as a microfluidic channel. The nucleic acids are in flow (in movement) throughout the analysis, and thus the methods provided herein are distinguished from “static” methods that fix parent nucleic acids and/or their digested fragments. The channels or other suitable devices are filled with liquids and not gels (i.e., it is a gel-free or sieve-free method). The methods do not require any form of surface derivatization of the channel walls. The methods provided herein also detect nucleic acids in a coiled state. That is, for the methods provided herein, it is important for the nucleic acids to be stretched early on in the process and while they are cleaved by RE. Beyond such cleavage, including while they are being detected and their fluorescence is being measured, the nucleic acids may be and typically are in a relaxed or coiled state.

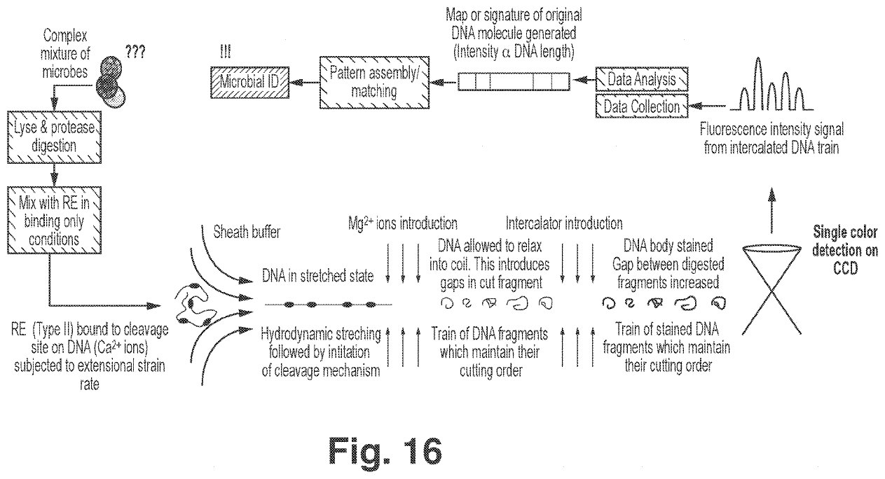

Another embodiment of the disclosure is provided in FIG. 16. This Figure illustrates the various steps starting from a sample having a complex mixture of microbes (or pathogens), lysis and DNA harvest from such sample, binding of the harvested DNA with RE in a binding condition (Ca²⁺ and no Mg²⁺ or Mn^{2±}), hydrodynamic stretching of the DNA and exposure of the DNA to the cleaving condition (e.g., introduction of Mg²⁺ ions), allowing the DNA to relax into a coiled state, staining the DNA with intercalator, optionally increasing the distance between digested fragments, detection of the fragments using for example a single color detector CCD, data collection and analysis, creation of a map or signature of the parent DNA (based on the proportional relationship between fluorescence intensity and DNA length), pattern assembly/matching (e.g., arranging various fragments relating to each other, including with overlap between fragments), and optionally identification of one or more microbes in the original complex sample.

According to one aspect, in some embodiments, the methods herein can be combined with polymerase chain reaction (“PCR”). The inventor has appreciated that integrating the methods of manipulating a nucleic acid as described herein with known PCR methods may, in some embodiments, help to reduce erroneous results in PCR, such as false positive and false negative rates. In some embodiments, the methods described herein may aid selection of target sequence based primer probes for PCR.

In some embodiments, a sample may be passed through one of the devices described above, and then at least a portion of the same sample may be used for PCR.

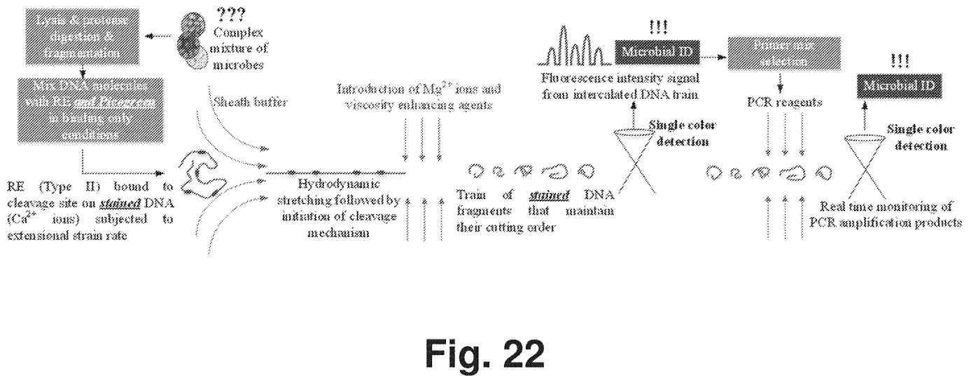

FIG. 22 depicts one schematic of an illustrative method that integrates one of the methods described herein with a PCR process. In this illustrative method, a sample goes through a stretching process as described above and results in DNA fragments that are maintained in their cutting order. These DNA fragments are then used in a PCR process.

### Sample Preparation

The samples being tested may be manipulated in a number of ways. For example, the samples may be washed and spun in order to concentrate cells. In some examples, the samples need not be washed and/or the cells need not be concentrated. Cells within the samples may be cultured prior to nucleic acid harvest or they may be used directly without in vitro culture.

Cells are then lysed using any variety of methods. The resultant cell lysate is protease-treated, and the nucleic acids are released. The nucleic acids may then be sheared, for example by hydrodynamic shearing, for a particular period of time in order to produce parent nucleic acids of about the same size, and optionally in the range of 20-500 kbp (kilo base pairs). Alternatively, the nucleic acids may be cut with a rare cutter RE in order to obtain fragments of a particular length. Examples of such rare cutters include but are not limited to NotI, XbaI and ApaI. The fragments that result after digestion with such rare cutters are mostly in the size range of 20-700 kbp.

Sequence-Specific Endonucleases including Restriction Enzymes (REs)

The sequence-specific endonuclease is a nuclease that binds to and cleaves a nucleic acid such as a DNA in a sequence-dependent manner. An example of a sequence-specific endonuclease is a restriction enzyme (RE).

The RE may be a type I RE, a type II RE, a type III RE or a type IV RE. Reference can be made to Pingoud et al. for the details of each RE type. (Pingoud et al. CMLS, Cell. Mol. Life Sci. 62: 685-707, 2005. In some embodiments, the RE is a type II RE. Type II RE tend to cleave within or close to their recognition site and do not require ATP (as do Types I and III) or GTP (as does Type IV). Most type II RE utilize Mg²⁺ for cleavage. Type II REs cleave DNA at defined sites that are 4-8 bp (base pairs) in length. Most type II REs belong to the PD . . . D/ExK family of REs.

Examples of type II RE include but are not limited to ApaI, BamHI, Bgll, Bglll, EcoRI, EcoRV, Muni, PvuII, Haelll, HinPI, Notl, Pmel, SmaI and Eagl. Examples of PD . . . D/ExK REs include BamHI, BglII, BsoBI, Bse634I, Cfr10I, EcoRI, EcoRII, EcoRV, FokI, MunI, and NgoMIV.

In some embodiments, a combination of RE is used. An example of such a combination is ApaI, BamHI and SmaI. The choice of REs may in some instances be governed by the degree of cutting that is desired. In some embodiments, if a combination of REs is used, the combination may include a low frequency (or rare) cutter, a medium frequency cutter, and a high frequency cutter.

It is to be understood that if more than one RE is used to analyze the DNA, the REs may be used simultaneously (i.e., a mixture of such RE is incubated with the DNA) or they may be used individually but in parallel.

### Conditions

The sample fluid may be virtually any fluid through which nucleic acids can travel. At a minimum, typically, it may be an aqueous solution. It may optionally comprise buffering agents, salts with the provisos provided herein, preservatives, and the like.

RE binding relies on diffusion of the nucleic acids and RE in order for RE to find and bind to their binding/cleavage site. This process usually takes about 15-60 minutes to complete. The methods described herein take advantage of the fact that the sequence-specific binding and cleavage activities of REs can be separated in time. That is, under certain conditions, it is possible for a RE to bind to a parent nucleic acid in a sequence-specific manner without cleaving the nucleic acid. The conditions can then be changed in order to cause cleavage by the RE.

REs require a sufficient concentration of magnesium ions (Mg²⁺) or manganese ions (Mn²⁺) to cleave a nucleic acid. In the methods provided herein, the parent nucleic acids are contacted with REs in the absence of Mg²⁺ and Mn²⁺ (or in insufficient amounts or concentrations of one or both of these ions). Under such conditions, the REs are able to bind to the nucleic acids in a sequence-specific manner but they are not able to cleave the nucleic acids. The binding sheath fluid may contain calcium ions (Ca²⁺), although this is not an absolute requirement. The RE may be modified versions of naturally occurring RE, that are able to bind and cut under different conditions. Thus, the nature of the binding and cleaving conditions may vary depending on the nature of the RE used. Conditions that allow REs to bind but not cleave nucleic acids are referred to herein as binding conditions. Conditions that allow nucleic acid-bound REs to cleave nucleic acids are referred to herein as cleaving conditions. The mechanisms by which REs bind in the absence of Mg²⁺ and Mn²⁺ and in the presence of Ca²⁺ and cleave in the presence of Mg²⁺ and/or Mn²⁺ are described in detail in Pingoud et al. CMLS, Cell. Mol. Life Sci., 62:685-707, 2005. The distinction between binding and cleaving conditions and RE activities have also been described by Belkebir and Azeddoug, Microbiol. Res. 168:99-105, 2013 for SepMI and EhoI REs.

Thus it will be understood that in various aspects of this disclosure the nucleic acids are contacted with a plurality of REs (of identical type) in a binding condition thereby allowing the REs to bind to the parent nucleic acids, and then the nucleic acids with REs bound thereto are placed in a cleaving condition thereby allowing the REs to cleave the parent nucleic acids. Once the REs cleave the parent nucleic acids, the REs will dissociate from the nucleic acid.

The binding condition may be a condition that comprises a Mg²⁺ concentration or a Mn²⁺ concentration or a Mg²⁺/Mn²⁺ combined concentration that does not allow cleavage of a nucleic acid. The binding condition may be a condition that lacks Mg²⁺, that lacks Mn²⁺, or that lacks Mg²⁺ and Mn²⁺. The binding condition may be a condition that comprises Ca²⁺ only and no Mg²⁺ or Mn²⁺ ions. Most Type 2 REs inherently show binding only activity in conditions where Ca²⁺ only is present without any Mg²⁺/Mn²⁺ ions.

Some embodiments of this disclosure provide for the use of a combination of RE simultaneously. In these embodiments, the binding and cutting conditions are chosen such that each of the two or more RE bind and cut the DNA with about equal efficiency. Cutting buffers to be used for two or more RE are known in the art and some are available commercially. For example, the Cutsmart® Buffer, available from New England BioLabs, comprises 50 mM Potassium Acetate, 20 mM Tris-acetate, 10 mM Magnesium Acetate, 100 μg/ml BSA, and has a pH 7.9 at 25° C. This buffer provides a suitable cutting condition for most RE, and thus may be regarded as a universal cutting buffer. Cutting conditions of the methods provided herein may similarly comprise about 50 mM Potassium Acetate, 20 mM Tris-acetate, about 10 mM Magnesium Acetate, about 100 μg/ml BSA, and have about a pH 7.9 at about 25° C., as an example. Binding conditions, on the other hand, may be similar to these cutting conditions except that the Magnesium Acetate would be substituted with a calcium salt such as but not limited to Calcium Acetate. Thus, in some embodiments, the binding conditions comprise 50 mM Potassium Acetate, 20 mM Tris-acetate, 10 mM Calcium Acetate, 100 μg/ml BSA, and has a pH 7.9 at 25° C. This buffer provides a suitable binding condition (no cutting) for most RE, and thus may be regarded as a universal binding buffer.

The cleaving condition may be a condition that comprises a Mg²⁺ concentration that allows cleavage of a nucleic acid. The cleaving condition may be a condition that comprises a Mg²⁺ concentration in the range of 5-100 mM. A concentration of about 10 mM Mg²⁺ may be used to activate the cleavage activity of most REs. The cleaving condition may be a condition that comprises a Mn²⁺ concentration that allows cleavage of a nucleic acid. The cleaving condition may be a condition that comprises a Mn²⁺ concentration in the range of 5-100 mM. A concentration of about 10 mM Mn²⁺ may be used to activate the cleavage activity of most REs.

### Intercalators

The nucleic acid fragments are labeled with intercalators. As used herein, intercalators are compounds that bind nucleic acids in a substantially sequence-independent manner. Examples include phenanthridines and acridines (e.g., ethidium bromide, propidium iodide, hexidium iodide, dihydroethidium, ethidium homodimer-1 and -2, ethidium monoazide, and ACMA); indoles and imidazoles (e.g., Hoechst 33258, Hoechst 33342, Hoechst 34580 and DAPI); cyanine dyes such as SYTOX Blue, SYTOX Green, SYTOX Orange, POPO-1, POPO-3, YOYO-1, YOYO-3, TOTO-1, TOTO-3, JOJO-1, LOLO-1, BOBO-1, BOBO-3, PO-PRO-1, PO-PRO-3, BO-PRO-1, BO-PRO-3, TO-PRO-1, TO-PRO-3, TO-PRO-5, JO-PRO-1, LO-PRO-1, YO-PRO-1, YO-PRO-3, PicoGreen®, OliGreen®, RiboGreen®, SYBR™ Gold, SYBR™ Green I, SYBR™ Green II, SYBR™ DX, SYTO-40, -41, -42, -43, -44, -45 (blue), SYTO-13, -16, -24, -21, -23, -12, -11, -20, -22, -15, -14, -25 (green), SYTO-81, -80, -82, -83, -84, -85 (orange), SYTO-64, -17, -59, -61, -62, -60, -63 (red); and miscellaneous stains such as acridine orange (also capable of intercalating), 7-AAD, actinomycin D, LDS751, and hydroxystilbamidine. All of the aforementioned nucleic acid stains are commercially available from suppliers such as Molecular Probes, Inc.

In some embodiments, the intercalator is PicoGreen® or SYBR™ Green, both of which are commercially available. These and other intercalators may be used to label samples having a wide range of DNA concentrations, and thus such intercalators are characterized as being less sensitive to DNA concentration, while still being sufficiently fluorescent. When such intercalators are used, it is not absolutely required that the DNA concentration of each sample be known and/or standardized. In some embodiments, such intercalators may be used in a micromolar range, including from example from about 0.5 to 1.5 micromolar, or about 0.7 to 1.3 micromolar, or about 0.7 to 1 micromolar. In some embodiments, the intercalator is PicoGreen® and it is used at a concentration of about 0.8 micromolar. PicoGreen® reportedly can be used to stain DNA concentrations in the range of 0.5-10,000 ng/ml stoichiometrically. Larson et al. Cytometry, 2000, 41:203-208.

A further advantage of certain intercalators including PicoGreen® and SYBER™Green is their ability to bind to DNA without inducing structural changes in the bound DNA. Schafer et al. Single Mol. 2000, 1:33-40. Since the intercalator does not induce structural changes in the DNA, it also does not impact binding of the RE to the DNA. Accordingly, labeling of a nucleic acid sample with such an intercalator can occur before and/or during binding of the RE.

Still a further advantage of certain intercalators including PicoGreen® and SYBER™Green is the ability to function even in relatively high salt conditions such as those used in RE digestion protocols.

The ability to determine size of nucleic acids based on fluorescent signal from bound intercalators is described by Huang et al. Nucleic Acids Research, 24(21):4202-4209, 1996.

### Applications

The methods provided herein can be used to perform detailed genomic DNA mapping of cultured cells. The cells may have been provided as a sample such as a microbiome sample from a normal (healthy) subject or from a subject having or suspected of having an infection. The microbiome sample may be an environmental sample such as a soil sample or a water sample. The sample may a bodily sample such as a urine sample, a blood sample, a sputum sample, or a bowel sample. In the case of a blood sample, the sample may be analyzed for the presence of rare circulating cells such as cancer cells. The sample may be a food sample or a sample derived from a food source. The sample may be a seed sample or a plant that is suspected of being genetically engineered.

The methods may be used to identify one or more or all known pathogens in a sample. In this embodiment, it is contemplated that a genomic map is created and compared to existing genomic maps of known pathogens. The existing genomic maps may be maps generated using other technologies such as PFGE or they may be theoretical maps obtained from knowledge of sequence specificity of an RE and sequence data collected from whole genome sequencing of particular pathogens. Alternatively, the existing genomic maps may be generated using the methodology of this disclosure as applied to pure samples of known pathogens.

The methods may be used to identify variants or mutants of known pathogens. In this latter aspect, it is contemplated that a genomic map is created and compared to existing genomic maps of known pathogens. Newly generated genomic maps that are closely related to existing genomic maps may represent mutants of known pathogens.

The genomic maps obtained using the methods described herein may be used instead of or as a supplement to existing genomic maps generated using other technologies such as but not limited to PFGE.

The genomic maps can then be used to assemble sequence information that is obtained from short read sequences, as is typically obtained from next-generation sequencing technologies such as but not limited to Illumina.

Each patent, patent application and reference cited herein is incorporated by reference in its entirety.

### ADDITIONAL EMBODIMENTS

The following are additional embodiments embraced by this disclosure.

### Embodiment 1

A device for manipulating a polymer in a fluid sample, the device comprising: a sample fluid inlet; a channel in fluid communication with the sample fluid inlet, the channel having a focusing region, a cutting region, relaxation region, and a detection region, the focusing region having a converging width shape; the cutting region having a converging width shape; and the relaxation region having a first portion having a converging width shape and a second portion having a diverging width shape, the converging width shape of the first portion of the relaxation region being a different shape than the converging width shape of the cutting region.

### Embodiment 2

The device of embodiment 1, wherein the converging width shape of the first portion of the relaxation region has a sharper rate of convergence than the converging width shape of the cutting region.

### Embodiment 3

The device of embodiment 1, wherein the converging width shape of the focusing region is a different shape than the converging width shape of the cutting region.

### Embodiment 4

The device of embodiment 3, wherein the converging width shape of the focusing region has a sharper rate of convergence than the converging width shape of the cutting region.

### Embodiment 5

The device of embodiment 1, wherein the converging width shape of the first portion of the relaxation region is a different shape than the converging width shape of the focusing region.

### Embodiment 6

The device of embodiment 1, further comprising a pair of supplementary inlets in fluid communication with the channel, the pair of supplementary inlets symmetrically opposing one another on either side of the channel.

### Embodiment 7

The device of embodiment 6, wherein the supplementary inlets are connected to the channel at the focusing region of the channel.

### Embodiment 8

The device of embodiment 6, further comprising a second pair of supplementary inlets in fluid communication with the channel, the pair of supplementary inlets symmetrically opposing one another on either side of the channel.

### Embodiment 9

The device of embodiment 1, wherein one of the supplementary inlets is positioned above the channel and the other supplementary inlet is positioned below the channel.

### Embodiment 10

The device of embodiment 1, wherein a width of the converging width shape of the focusing region is proportional to a function of 1/x, where x is distance along the focusing region of the channel.

### Embodiment 11

The device of embodiment 1, wherein the converging width shape of the cutting region has a linearly decreasing width.

### Embodiment 12

The device of embodiment 1, wherein the converging width shape of the first portion of the relaxation region has a linearly decreasing width.

### Embodiment 13

The device of embodiment 1, wherein the diverging shape of the second portion of the relaxation region has a linearly increasing width.

### Embodiment 15

The device of embodiment 1, wherein the converging width shape of the cutting region has a constant strain rate profile.

### Embodiment 16

The device of embodiment 1, wherein the converging width shape of the first portion of the relaxation region has a constant acceleration profile.

### Embodiment 17

A method for manipulating a nucleic acid in flow comprising: stretching a parent nucleic acid as the parent nucleic acid moves through a channel; digesting the parent nucleic acid, in flow, with a sequence-specific endonuclease to generate a plurality of digested fragments; and maintaining the digested fragments in a linear arrangement in flow that represents the order of the fragments in the parent nucleic acid.

### Embodiment 18

The method of embodiment 17, further comprising: allowing the digested fragments to relax to cause gaps to form between the digested fragments and to cause the digested fragments to coil.

### Embodiment 19

The method of embodiment 17, wherein stretching the parent nucleic acid comprises flowing the parent nucleic acid through a focusing region of the channel having a converging width shape.

### Embodiment 20

The method of embodiment 18, wherein allowing the digested fragments to relax comprises flowing the digested fragments through a relaxation region of the channel having a first portion with a converging width shape and a second portion with a diverging width shape.

### Embodiment 21

The method of embodiment 17, wherein the method is non-electrophoretic.

### Embodiment 22

The method of embodiment 17, wherein the nucleic acid is stretched and moved by hydrodynamic force.

### Embodiment 23

A method for obtaining sequence information from a nucleic acid comprising: incubating a parent nucleic acid with a plurality of restriction enzymes under conditions that allow the restriction enzymes to bind to but not cleave the nucleic acid; elongating the parent nucleic acid with bound restriction enzymes while in flow; altering the conditions sufficiently to cause the bound restriction enzymes to cleave the parent nucleic acid, thereby creating a plurality of digested fragments linearly arranged in flow; staining the digested fragments with an intercalator while maintaining the position of each relative to the other digested fragments; and measuring fluorescence intensity of each digested fragment individually in a sequential manner, wherein the fluorescence intensity and detection order of the digested fragments, together with the sequence specificity of the restriction enzyme yield a map of the parent nucleic acid.

### EXAMPLES

### Example 1. Binding and Cutting Conditions

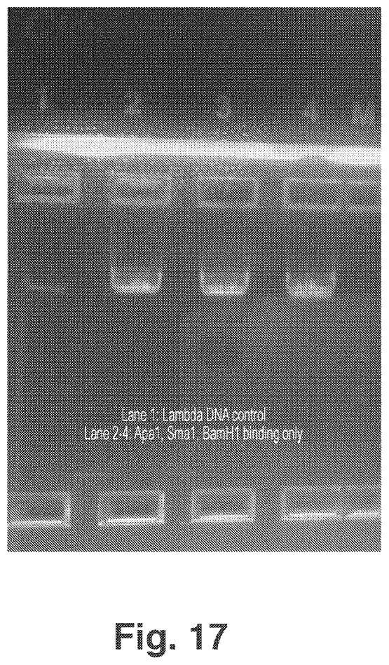

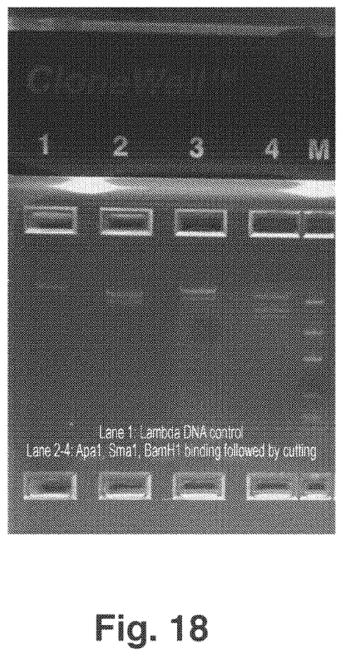

Lambda DNA and RE were mixed in binding (only) buffer conditions (i.e., 50 mM Potassium Acetate, 20 mM Tris-acetate, 10 mM Calcium Acetate, 100 μg/ml BSA, and has a pH 7.9 at 25° C.). FIG. 17 confirms that in these conditions no cutting of DNA molecule occurs as seen from gel electrophoresis. Lane 1 contains lambda DNA control, and lanes 2-4 contain ApaI, SmaI and BamHI respectively. As a control, a portion of the DNA/RE mixture was spiked with 10× Cutsmart® Buffer for a final concentration of 1× Cutsmart® Buffer and 0.1× binding (only) buffer. This produced the expected digestion maps on gel electrophoresis as shown in FIG. 18. Lane 1 contains lambda DNA control, and lanes 2-4 contain ApaI, SmaI and BamHI respectively, wherein the samples in lanes 2-4 were first incubated with binding only buffer and then incubated with cutting buffer (i.e., the spiked buffer described above).

### Example 2. Intercalator Staining Under Binding Conditions

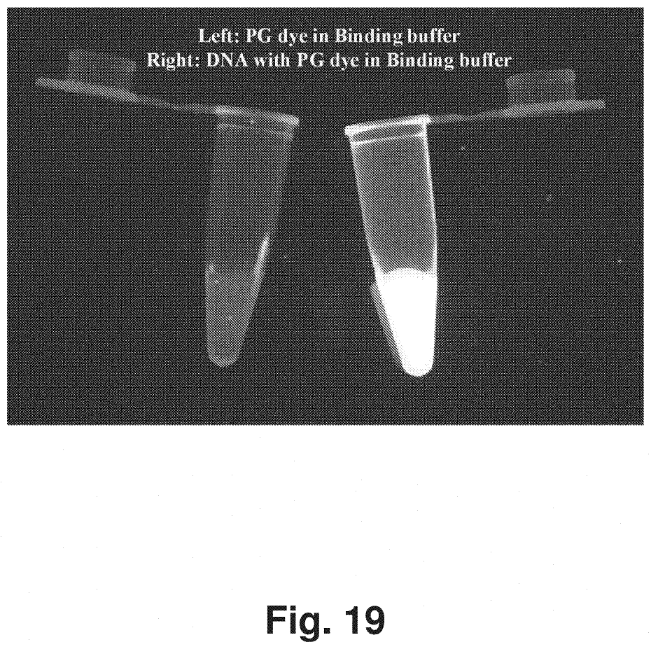

PicoGreen® staining of a DNA-RE mixture was performed in binding (only) buffer conditions. FIG. 19 shows PicoGreen® stained DNA in the presence of binding only buffer (right tube) and PicoGreen® and binding only buffer control (left tube). PicoGreen® provides enhanced fluorescence intensity in the presence of DNA in the binding only buffer condition. The tubes are illuminated by blue light and an orange filter is used to filter fluorescence signal onto an iPhone camera.

### REFERENCES

1. van den Broek, Bram, Maarten C. Noom, and Gijs J L Wuite. “DNA-tension dependence of restriction enzyme activity reveals mechanochemical properties of the reaction pathway.” Nucleic acids research 33.8 (2005): 2676-2684.
2. Robinson, Clifford R., and Stephen G. Sligar. “Heterogeneity in molecular recognition by restriction endonucleases: osmotic and hydrostatic pressure effects on BamHI, Pvu II, and EcoRV specificity.” Proceedings of the National Academy of Sciences 92.8 (1995): 3444-3448.
3. Robinson, Clifford R., and Stephen G. Sligar. “Changes in solvation during DNA binding and cleavage are critical to altered specificity of the EcoRI endonuclease.” Proceedings of the National Academy of Sciences 95.5 (1998): 2186-2191.
4. Wenner, Jay R., and Victor A. Bloomfield. “Crowding effects on EcoRV kinetics and binding.” Biophysical journal 77.6 (1999): 3234-3241.
5. Smith, Douglas E., Hazen P. Babcock, and Steven Chu. “Single-polymer dynamics in steady shear flow.” Science 283.5408 (1999): 1724-1727.
6. Smith, Douglas E., and Steven Chu. “Response of flexible polymers to a sudden elongational flow.” Science 281.5381 (1998): 1335-1340.
7. Larson, Jonathan W., et al. “Single DNA molecule stretching in sudden mixed shear and elongational microflows.” Lab on a Chip 6.9 (2006): 1187-1199.
8. Riehn, Robert, et al. “Restriction mapping in nanofluidic devices.” Proceedings of the National Academy of Sciences of the United States of America 102.29 (2005): 10012-10016.
9. Wilson, Gary A., and Frank E. Young. “Isolation of a sequence-specific endonuclease (BamI) from Bacillus amyloliquefaciens H.” Journal of molecular biology 97.1 (1975): 123-125.
10. Reisner, Walter, et al. “Statics and dynamics of single DNA molecules confined in nanochannels.” Physical Review Letters 94.19 (2005): 196101.
11. Ellis R J: Macromolecular crowding: an important but neglected aspect of the intracellular environment. Current Opinion in Structural Biology. 2001, 11 (1): 114-119.
12. Zimmerman S B, Harrison B: Macromolecular crowding increases binding of DNA polymerase to DNA: an adaptive effect. Proc Natl Acad Sci. 1987, 84 (7): 1871-1875.
13. Wenner J R, Bloomfield V A: Crowding Effects on EcoRV Kinetics and Binding. Biophysical Journal. 1999, 77 (6): 3234-3241.
14. Minton A P: The influence of macromolecular crowding and macromolecular confinement on biochemical reactions in physiological media. The Journal of Biological Chemistry. 2001, 276: 10577-10580.
15. Sasaki Y, Miyoshi D, Sugimoto N: Regulation of DNA nucleases by molecular crowding. Nucleic Acids Research. 2007, 35 (12): 4086-4093.
16. Cheng, Nian-Sheng. “Formula for the viscosity of a glycerol-water mixture.” Industrial & engineering chemistry research 47.9 (2008): 3285-3288.
17. Conlan, L. H., et al. “Modulating restriction endonuclease activities and specificities using neutral detergents.” Biotechniques 27.5 (1999): 955-960.

1. A device for manipulating a polymer in a fluid sample, the device comprising:
a sample fluid inlet;
a channel in fluid communication with the sample fluid inlet, the channel having a focusing region, a cutting region, a relaxation region, and a detection region,
the focusing region including a pair of supplementary inlets in fluid communication with the channel, the pair of supplementary inlets opposing one another on either side of the channel,
the cutting region having a converging width shape.

2. The device of claim 1, wherein the relaxation region has a first portion having a converging width shape and a second portion having a diverging width shape.

3. The device of claim 2, wherein the converging width shape of the first portion of the relaxation region has a sharper rate of convergence than the converging width shape of the cutting region.

4. The device of claim 1, wherein at least a portion of the focusing region has a converging width shape.

5. (canceled)

6. The device of claim 4, wherein the converging width shape of the focusing region has a sharper rate of convergence than the converging width shape of the cutting region.

7. The device of claim 2, wherein at least a portion of the focusing region has a converging width shape, and wherein the converging width shape of the first portion of the relaxation region is a different shape than the converging width shape of the focusing region.

8. The device of claim 1, wherein the supplementary inlets are connected to the channel at the focusing region of the channel.

9. (canceled)

10. (canceled)

11. The device of claim 4, wherein a width of the converging width shape of the focusing region is proportional to a function of 1/x, where x is distance along the focusing region of the channel.

12. The device of claim 1, wherein the converging width shape of the cutting region has a linearly decreasing width.

13. The device of claim 2, wherein the converging width shape of the first portion of the relaxation region has a linearly decreasing width.

14. The device of claim 2, wherein the diverging shape of the second portion of the relaxation region has a linearly increasing width.

15. The device of claim 1, wherein the converging width shape of the cutting region has a constant strain rate profile.

16. The device of claim 2, wherein the converging width shape of the first portion of the relaxation region has a constant acceleration profile.

17. (canceled)

18. (canceled)

19. A method for manipulating a nucleic acid in flow comprising:
stretching a parent nucleic acid as the parent nucleic acid moves through a channel;
digesting the parent nucleic acid, in flow, with a sequence-specific endonuclease to generate a plurality of digested fragments; and
maintaining the digested fragments in a linear arrangement in flow that represents the order of the fragments in the parent nucleic acid.

20. The method of claim 19, further comprising:
allowing the digested fragments to relax to cause gaps to form between the digested fragments and to cause the digested fragments to coil.

21. The method of claim 19, wherein stretching the parent nucleic acid comprises flowing the parent nucleic acid through a focusing region of the channel having a pair of supplementary inlets in fluid communication with the channel, the pair of supplementary inlets opposing one another on either side of the channel.

22. The method of claim 21, further comprising introducing viscosifying agents into the channel through the pair of supplementary inlets.

23. (canceled)

24. The method of claim 20, wherein allowing the digested fragments to relax comprises flowing the digested fragments through a relaxation region of the channel having a first portion with a converging width shape and a second portion with a diverging width shape.

25. (canceled)

26. (canceled)

27. (canceled)

28. A method for obtaining sequence information from a nucleic acid comprising:
incubating a parent nucleic acid with a plurality of restriction enzymes under conditions that allow the restriction enzymes to bind to but not cleave the nucleic acid;
elongating the parent nucleic acid with bound restriction enzymes while in flow;
altering the conditions sufficiently to cause the bound restriction enzymes to cleave the parent nucleic acid, thereby creating a plurality of digested fragments linearly arranged in flow;
staining the digested fragments with an intercalator while maintaining the position of each relative to the other digested fragments; and
measuring fluorescence intensity of each digested fragment individually in a sequential manner,
wherein the fluorescence intensity and detection order of the digested fragments, together with the sequence specificity of the restriction enzyme yield a map of the parent nucleic acid.

29. A method for obtaining sequence information from a nucleic acid comprising:
incubating a parent nucleic acid with a plurality of restriction enzymes under conditions that allow the restriction enzymes to bind to but not cleave the parent nucleic acid;
staining the parent nucleic acid having bound restriction enzymes with an intercalator;
elongating the parent stained nucleic acid with bound restriction enzymes while in flow;
altering the conditions sufficiently to cause the bound restriction enzymes to cleave the parent nucleic acid, thereby creating a plurality of digested fragments linearly arranged in flow; and
measuring fluorescence intensity of each digested fragment individually in a sequential manner,
wherein the fluorescence intensity and detection order of the digested fragments, together with the sequence specificity of the restriction enzyme yield a map of the parent nucleic acid.
